(19) United States
(12) Patent Application Publication
(10) Pub. No.: US 2022/0110917 A1
(43) Pub. Date: Apr. 14, 2022

(54) USE OF GABOXADOL, GANAXOLONE AND ALLOPREGNANOLONE TO TREAT MOVEMENT DISORDERS

(71) Applicant: Ovid Therapeutics Inc., New York, NY (US)

(72) Inventor: Matthew DURING, Darien, CT (US)

(21) Appl. No.: 17/558,195

(22) Filed: Dec. 21, 2021

Related U.S. Application Data
(62) Division of application No. 16/674,601, filed on Nov. 5, 2019.
(60) Provisional application No. 62/755,674, filed on Nov. 5, 2018.

(51) Int. Cl. A61K 31/4162; A61K 31/573

(52) U.S. Cl. CPC A61K 31/4162; A61K 31/573

(57) ABSTRACT

Use of gaboxadol, allopregnanolone, or ganaxolone, or a pharmaceutical salt of any of the foregoing, for the treatment of cervical dystonia, multiple system atrophy, myoclonus, progressive supranuclear palsy, restless legs syndrome, Wilson's disease, ataxia, chorea, or dystonia in a subject in need thereof.

### CROSS-REFERENCE TO RELATED APPLICATIONS

This application is a divisional application of U.S. Ser. No. 16/674,601, filed Nov. 5, 2019, which claims benefit and priority to U.S. Provisional Application No. 62/755,674, filed Nov. 5, 2018, which are both incorporated herein by reference in their entireties.

### FIELD OF THE INVENTION

Methods of treating movement disorders with gaboxadol, ganaxolone and allopregnanolone or pharmaceutical salts thereof are provided.

### BACKGROUND

Movement disorders are conditions that cause problems with movement such as increased movement, or decreased or slow movement. They may result in spastic movement. Movement disorders can involve abnormalities in muscle tone and motor control. They can encompass neurodegenerative disorders and neurodevelopmental disorders. The following conditions are movement disorders.

Cervical dystonia causes long-lasting contractions (spasms) or intermittent contractions of the neck muscles, causing the neck to turn in different ways. Multiple system atrophy is an uncommon, progressive neurological disorder that affects many brain systems. Multiple system atrophy causes a movement disorder, such as ataxia or parkinsonism. It can also cause low blood pressure and impaired bladder function. Myoclonus causes lightning-quick jerks of a muscle or a group of muscles. Progressive supranuclear palsy is a rare neurological disorder that causes problems with walking, balance and eye movements. It may resemble Parkinson's disease but is a distinct condition. Restless legs syndrome causes unpleasant, abnormal feelings in the legs while relaxing or lying down, often relieved by movement. Wilson's disease is a rare inherited disorder that causes excessive amounts of copper to build up in the body, causing movement disorders. Ataxia is characterized by uncoordinated or clumsy balance, speech or limb movements. Chorea is characterized by repetitive, brief, irregular, somewhat rapid, involuntary movements that typically involve the face, mouth, trunk and limbs. Dystonia involves sustained involuntary muscle contractions with twisting, repetitive movements. Dystonia may affect the entire body (generalized dystonia) or one part of the body (focal dystonia).

Gaboxadol (4,5,6,7-tetrahydroisoxazolo [5,4-c] pyridine-3-ol) (THIP)) is described in EP Patent No. 0000338, in EP Patent No. 0840601, and in U.S. Pat. Nos. 4,278,676, 4,362,731, 4,353,910, and WO 2005/094820. Gaboxadol is a selective GABA_{A} receptor agonist with a preference for δ-subunit containing GABA_{A} receptors. In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia but failed to show significant effects in sleep onset and sleep maintenance in a three-month efficacy study. Additionally, patients with a history of drug abuse who received gaboxadol experienced a steep increase in psychiatric adverse events. As a result of these negative results the development of gaboxadol was terminated at that time.

Allopregnanolone, also known as 3α, 5α-tetrahydroprogesterone (THP) is a neuroactive steroid derived from progesterone. In contrast to progesterone, allopregnanolone does not bind to progesterone receptors, but is rather a positive modulator of γ-aminobutyric acid (GABA)_{A} receptors. Allopregnanolone is rapidly redistributed from the brain, and the first compartment half-life is very short. Turkmen et al., British Journal of Pharmacology (2011) 162(2) 311-327. The half-life of allopregnanolone is about 35 minutes. Budimirovic, Neurotherapeutics (2017) 14:1070-1072. Allopregnanolone has been in clinical trials for treatment of traumatic brain injury, mild cognitive impairment due to Alzheimer's disease, and Fragile X Associated Tremor/Ataxia syndrome (FXTAS).

Ganaxolone is the 3β-methylated synthetic analog of allopregnanolone and is also classified as a neurosteroid. Ganaxolone is a positive allosteric modulator of GABA-A receptors.

The Tₘₐₓ of ganaxolone following oral administration in suspension is 1.2-2.5 h, with a plasma half-life of approximately 20 h. Nohria and Giller, Neurotherapeutics (2007) January; 4(1):102-105. Ganaxolone has been in clinical trials for post-partum depression, PCDH19 Female Pediatric Epilepsy, CDKL5 Deficiency Disorder, Fragile-X syndrome and posttraumatic stress disorder.

### SUMMARY

Methods are provided for treatment of movement disorders including cervical dystonia, multiple system atrophy, myoclonus, progressive supranuclear palsy, restless legs syndrome, Wilson's disease, ataxia, chorea, or dystonia. Methods of treating the foregoing movement disorders are provided which include administering to a patient in need thereof gaboxadol, ganaxolone or allopregnanolone or a pharmaceutically acceptable salt of any of the foregoing.

Methods of treating cervical dystonia, multiple system atrophy, myoclonus, progressive supranuclear palsy, restless legs syndrome, Wilson's disease, ataxia, chorea, or dystonia include administering gaboxadol or a pharmaceutically acceptable salt thereof to a patient in need thereof to provide improvement in one or more symptoms of the cervical dystonia, multiple system atrophy, myoclonus, progressive supranuclear palsy, restless legs syndrome, Wilson's disease, ataxia, chorea, or dystonia. Pharmaceutical compositions including gaboxadol or a pharmaceutically acceptable salt thereof are provided for use in treating cervical dystonia, multiple system atrophy, myoclonus, progressive supranuclear palsy, restless legs syndrome, Wilson's disease, ataxia, chorea, or dystonia. Methods of treating cervical dystonia, multiple system atrophy, myoclonus, progressive supranuclear palsy, restless legs syndrome, Wilson's disease, ataxia, chorea, or dystonia include administering a pharmaceutical composition including gaboxadol or a pharmaceutically acceptable salt thereof to a patient in need thereof. In embodiments, methods of treating cervical dystonia, multiple system atrophy, myoclonus, progressive supranuclear palsy, restless legs syndrome, Wilson's disease, ataxia, chorea, or dystonia include administering gaboxadol or a pharmaceutically acceptable salt thereof to a patient in need thereof to provide improvement in symptoms of the cervical dystonia, multiple system atrophy, myoclonus, progressive supranuclear palsy, restless legs syndrome, Wilson's disease, ataxia, chorea, or dystonia in the patient a day after administration of the gaboxadol or a pharmaceutically acceptable salt thereof.

Methods of treating cervical dystonia, multiple system atrophy, myoclonus, progressive supranuclear palsy, restless legs syndrome, Wilson's disease, ataxia, chorea, or dystonia are described herein which include administering to a patient in need thereof gaboxadol or a pharmaceutically acceptable salt thereof wherein the method provides an in vivo plasma profile including a Cₘₐₓ less than about 400 ng/ml and wherein the method provides improvement in the patient for more than 6 hours after administration of the gaboxadol or a pharmaceutically acceptable salt thereof. Methods of treating cervical dystonia, multiple system atrophy, myoclonus, progressive supranuclear palsy, restless legs syndrome, Wilson's disease, ataxia, chorea, or dystonia are described herein which include administering to a patient in need thereof a composition including gaboxadol or a pharmaceutically acceptable salt thereof wherein the composition provides an in vivo plasma profile including a Cₘₐₓ less than about 400 ng/ml and wherein the method provides improvement in the patient for more than 6 hours after administration of the gaboxadol or a pharmaceutically acceptable salt thereof. Methods of treating cervical dystonia, multiple system atrophy, myoclonus, progressive supranuclear palsy, restless legs syndrome, Wilson's disease, ataxia, chorea, or dystonia are described herein which include administering to a patient in need thereof gaboxadol or a pharmaceutically acceptable salt thereof wherein the method provides an in vivo plasma profile comprising a AUC₆₋₁₂ of less than about 900 ng·hr/ml and wherein the method provides improvement in the patient for more than 6 hours after administration of the gaboxadol or a pharmaceutically acceptable salt thereof. Methods of treating cervical dystonia, multiple system atrophy, myoclonus, progressive supranuclear palsy, restless legs syndrome, Wilson's disease, ataxia, chorea, or dystonia are described herein which include administering to a patient in need thereof a composition including gaboxadol or a pharmaceutically acceptable salt thereof wherein the composition provides an in vivo plasma profile comprising a AUC₆₋₁₂ of less than about 900 ng·hr/ml and wherein the composition provides improvement in the patient for more than 6 hours after administration of the gaboxadol or a pharmaceutically acceptable salt thereof. Methods of treating cervical dystonia, multiple system atrophy, myoclonus, progressive supranuclear palsy, restless legs syndrome, Wilson's disease, ataxia, chorea, or dystonia are described herein which include administering to a patient in need thereof a first pharmaceutical composition including gaboxadol or a pharmaceutically acceptable salt thereof and a second pharmaceutical composition including gaboxadol or a pharmaceutically acceptable salt thereof wherein the second pharmaceutical composition provides an in vivo plasma profile having a mean AUC_{0-∞} of at least 20% less than the first pharmaceutical composition.

Methods of treating cervical dystonia, multiple system atrophy, myoclonus, progressive supranuclear palsy, restless legs syndrome, Wilson's disease, ataxia, chorea, or dystonia include administering allopregnanolone or a pharmaceutically acceptable salt thereof to a patient in need thereof to provide improvement in one or more symptoms of the cervical dystonia, multiple system atrophy, myoclonus, progressive supranuclear palsy, restless legs syndrome, Wilson's disease, ataxia, chorea, or dystonia. Pharmaceutical compositions including allopregnanolone or a pharmaceutically acceptable salt thereof are provided for use in treating cervical dystonia, multiple system atrophy, myoclonus, progressive supranuclear palsy, restless legs syndrome, Wilson's disease, ataxia, chorea, or dystonia. Methods of treating cervical dystonia, multiple system atrophy, myoclonus, progressive supranuclear palsy, restless legs syndrome, Wilson's disease, ataxia, chorea, or dystonia include administering a pharmaceutical composition including allopregnanolone or a pharmaceutically acceptable salt thereof to a patient in need thereof. In embodiments, methods of treating cervical dystonia, multiple system atrophy, myoclonus, progressive supranuclear palsy, restless legs syndrome, Wilson's disease, ataxia, chorea, or dystonia include administering allopregnanolone or a pharmaceutically acceptable salt thereof to a patient in need thereof to provide improvement in symptoms of the cervical dystonia, multiple system atrophy, myoclonus, progressive supranuclear palsy, restless legs syndrome, Wilson's disease, ataxia, chorea, or dystonia in the patient a day after administration of the allopregnanolone or a pharmaceutically acceptable salt thereof. Methods of treating cervical dystonia, multiple system atrophy, myoclonus, progressive supranuclear palsy, restless legs syndrome, Wilson's disease, ataxia, chorea, or dystonia are described herein which include administering to a patient in need thereof allopregnanolone or a pharmaceutically acceptable salt thereof wherein the method provides improvement in the patient for more than 6 hours after administration of the allopregnanolone or a pharmaceutically acceptable salt thereof.

Methods of treating cervical dystonia, multiple system atrophy, myoclonus, progressive supranuclear palsy, restless legs syndrome, Wilson's disease, ataxia, chorea, or dystonia include administering ganaxolone or a pharmaceutically acceptable salt thereof to a patient in need thereof to provide improvement in one or more symptoms of the cervical dystonia, multiple system atrophy, myoclonus, progressive supranuclear palsy, restless legs syndrome, Wilson's disease, ataxia, chorea, or dystonia. Pharmaceutical compositions including ganaxolone or a pharmaceutically acceptable salt thereof are provided for use in treating cervical dystonia, multiple system atrophy, myoclonus, progressive supranuclear palsy, restless legs syndrome, Wilson's disease, ataxia, chorea, or dystonia. Methods of treating cervical dystonia, multiple system atrophy, myoclonus, progressive supranuclear palsy, restless legs syndrome, Wilson's disease, ataxia, chorea, or dystonia include administering a pharmaceutical composition including ganaxolone or a pharmaceutically acceptable salt thereof to a patient in need thereof. In embodiments, methods of treating cervical dystonia, multiple system atrophy, myoclonus, progressive supranuclear palsy, restless legs syndrome, Wilson's disease, ataxia, chorea, or dystonia include administering ganaxolone or a pharmaceutically acceptable salt thereof to a patient in need thereof to provide improvement in symptoms of the cervical dystonia, multiple system atrophy, myoclonus, progressive supranuclear palsy, restless legs syndrome, Wilson's disease, ataxia, chorea, or dystonia in the patient a day after administration of the ganaxolone or a pharmaceutically acceptable salt thereof. Methods of treating cervical dystonia, multiple system atrophy, myoclonus, progressive supranuclear palsy, restless legs syndrome, Wilson's disease, ataxia, chorea, or dystonia are described herein which include administering to a patient in need thereof ganaxolone or a pharmaceutically acceptable salt thereof wherein the method provides improvement in the patient for more than 6 hours after administration of the allopregnanolone or a pharmaceutically acceptable salt thereof.

### BRIEF DESCRIPTION OF THE DRAWINGS

FIG. 1 shows the arithmetic mean plasma concentration-time profiles of gaboxadol following single oral doses (2.5, 5, 10, 15, and 20 mg) as described in Example 1 with horizontal lines Δ indicating the change between 6 and 12 hours.

FIG. 2 shows the arithmetic mean plasma concentration-time profiles of gaboxadol following single oral doses (2.5, 5, 10, 15, and 20 mg) as described in Example 1.

### DETAILED DESCRIPTION

Described herein are methods and compositions for treating movement disorders such as cervical dystonia, multiple system atrophy, myoclonus, progressive supranuclear palsy, restless legs syndrome, Wilson's disease, ataxia, chorea, or dystonia. The methods and compositions described herein involve gaboxadol, ganaxolone and/or allopregnanolone or a pharmaceutically acceptable salt of any of the foregoing.

In embodiments, a method of treating cervical dystonia, multiple system atrophy, myoclonus, progressive supranuclear palsy, restless legs syndrome, Wilson's disease, ataxia, chorea, or dystonia may include administering to a patient in need thereof a pharmaceutical composition including gaboxadol or a pharmaceutically acceptable salt thereof. In embodiments, a method of treating cervical dystonia, multiple system atrophy, myoclonus, progressive supranuclear palsy, restless legs syndrome, Wilson's disease, ataxia, chorea, or dystonia may include administering to a patient in need thereof a pharmaceutical composition including allopregnanolone or a pharmaceutically acceptable salt thereof. In embodiments, a method of treating cervical dystonia, multiple system atrophy, myoclonus, progressive supranuclear palsy, restless legs syndrome, Wilson's disease, ataxia, chorea, or dystonia may include administering to a patient in need thereof a pharmaceutical composition including ganaxolone or a pharmaceutically acceptable salt thereof.

Many pharmaceutical products are administered as a fixed dose, at regular intervals, to achieve therapeutic efficacy. The duration of action is reflected by the product's plasma half-life. Gaboxadol is a selective GABA_{A} receptor agonist with a relatively short half-life (t½=1.5 h). The half-life of allopregnanolone is about 35 minutes. The half-life of ganaxolone is approximately 20 h. Since efficacy is often dependent on sufficient exposure within the central nervous system administration of CNS drugs with a short half-life may require frequent maintenance dosing.

Advantageously disclosed herein are methods of treating cervical dystonia, multiple system atrophy, myoclonus, progressive supranuclear palsy, restless legs syndrome, Wilson's disease, ataxia, chorea, or dystonia by administration of gaboxadol or pharmaceutically acceptable salt thereof. For example, in embodiments, methods of treating an cervical dystonia, multiple system atrophy, myoclonus, progressive supranuclear palsy, restless legs syndrome, Wilson's disease, ataxia, chorea, or dystonia are provided which include administering to a patient in need thereof a pharmaceutical composition including about 0.05 mg to about 75 mg of gaboxadol or pharmaceutically acceptable salt thereof, wherein the composition provides improvement for more than 6 hours after administration to the patient. In embodiments, methods of treating cervical dystonia, multiple system atrophy, myoclonus, progressive supranuclear palsy, restless legs syndrome, Wilson's disease, ataxia, chorea, or dystonia are provided which include administering to a patient in need thereof a pharmaceutical composition including about 0.05 mg to about 10 mg of allopregnanolone or a pharmaceutically acceptable salt thereof, wherein the composition provides improvement in cervical dystonia, multiple system atrophy, myoclonus, progressive supranuclear palsy, restless legs syndrome, Wilson's disease, ataxia, chorea, or dystonia for more than 6 hours after administration to the patient. In embodiments, methods of treating cervical dystonia, multiple system atrophy, myoclonus, progressive supranuclear palsy, restless legs syndrome, Wilson's disease, ataxia, chorea, or dystonia are provided which include administering to a patient in need thereof a pharmaceutical composition including about 0.05 mg to about 2000 mg of ganaxolone or a pharmaceutically acceptable salt thereof, wherein the composition provides improvement in cervical dystonia, multiple system atrophy, myoclonus, progressive supranuclear palsy, restless legs syndrome, Wilson's disease, ataxia, chorea, or dystonia for more than 6 hours after administration to the patient.

Methods of treating cervical dystonia, multiple system atrophy, myoclonus, progressive supranuclear palsy, restless legs syndrome, Wilson's disease, ataxia, chorea, or dystonia described herein include administering to a patient in need thereof about 0.05 mg to about 30 mg gaboxadol or a pharmaceutically acceptable salt thereof wherein the method provides improvement in one or more symptoms of cervical dystonia, multiple system atrophy, myoclonus, progressive supranuclear palsy, restless legs syndrome, Wilson's disease, ataxia, chorea, or dystonia. Methods of treating cervical dystonia, multiple system atrophy, myoclonus, progressive supranuclear palsy, restless legs syndrome, Wilson's disease, ataxia, chorea, or dystonia described herein include administering to a patient in need thereof about 0.05 mg to about 30 mg gaboxadol or a pharmaceutically acceptable salt thereof wherein the method provides improvement in one or more symptoms of cervical dystonia, multiple system atrophy, myoclonus, progressive supranuclear palsy, restless legs syndrome, Wilson's disease, ataxia, chorea, or dystonia a day after administering the gaboxadol or a pharmaceutically acceptable salt thereof.

Methods of treating cervical dystonia, multiple system atrophy, myoclonus, progressive supranuclear palsy, restless legs syndrome, Wilson's disease, ataxia, chorea, or dystonia described herein include administering to a patient in need thereof 0.05 mg to about 10 mg of allopregnanolone or a pharmaceutically acceptable salt thereof wherein the method provides improvement in one or more symptoms of cervical dystonia, multiple system atrophy, myoclonus, progressive supranuclear palsy, restless legs syndrome, Wilson's disease, ataxia, chorea, or dystonia. Methods of treating cervical dystonia, multiple system atrophy, myoclonus, progressive supranuclear palsy, restless legs syndrome, Wilson's disease, ataxia, chorea, or dystonia described herein include administering to a patient in need thereof about 0.05 mg to about 10 mg of allopregnanolone or a pharmaceutically acceptable salt thereof wherein the method provides improvement in one or more symptoms of cervical dystonia, multiple system atrophy, myoclonus, progressive supranuclear palsy, restless legs syndrome, Wilson's disease, ataxia, chorea, or dystonia a day after administering the allopregnanolone or a pharmaceutically acceptable salt thereof.

Methods of treating cervical dystonia, multiple system atrophy, myoclonus, progressive supranuclear palsy, restless legs syndrome, Wilson's disease, ataxia, chorea, or dystonia described herein include administering to a patient in need thereof about 0.05 mg to about 2000 mg of ganaxolone or a pharmaceutically acceptable salt thereof wherein the method provides improvement in one or more symptoms of cervical dystonia, multiple system atrophy, myoclonus, progressive supranuclear palsy, restless legs syndrome, Wilson's disease, ataxia, chorea, or dystonia. Methods of treating cervical dystonia, multiple system atrophy, myoclonus, progressive supranuclear palsy, restless legs syndrome, Wilson's disease, ataxia, chorea, or dystonia described herein include administering to a patient in need thereof about 0.05 mg to about 2000 mg of ganaxolone or a pharmaceutically acceptable salt thereof wherein the method provides improvement in one or more symptoms of cervical dystonia, multiple system atrophy, myoclonus, progressive supranuclear palsy, restless legs syndrome, Wilson's disease, ataxia, chorea, or dystonia a day after administering the ganaxolone or a pharmaceutically acceptable salt thereof.

In embodiments, methods of treating cervical dystonia, multiple system atrophy, myoclonus, progressive supranuclear palsy, restless legs syndrome, Wilson's disease, ataxia, chorea, or dystonia are described herein which include administering to a patient in need thereof gaboxadol or a pharmaceutically acceptable salt thereof wherein the method provides an in vivo plasma profile including a Cₘₐₓ less than about 400 ng/ml and wherein the method provides improvement in the patient for more than 6 hours after administration of the gaboxadol or a pharmaceutically acceptable salt thereof. In embodiments, methods of treating cervical dystonia, multiple system atrophy, myoclonus, progressive supranuclear palsy, restless legs syndrome, Wilson's disease, ataxia, chorea, or dystonia are described herein which include administering to a patient in need thereof gaboxadol or a pharmaceutically acceptable salt thereof wherein the method provides an in vivo plasma profile comprising a AUC₆₋₁₂ of less than about 900 ng ng·hr/ml and wherein the method provides improvement in the patient for more than 6 hours after administration of the gaboxadol or a pharmaceutically acceptable salt thereof. In embodiments, methods of treating cervical dystonia, multiple system atrophy, myoclonus, progressive supranuclear palsy, restless legs syndrome, Wilson's disease, ataxia, chorea, or dystonia are described herein which include administering to a patient in need thereof a first pharmaceutical composition comprising gaboxadol or a pharmaceutically acceptable salt thereof and a second pharmaceutical composition comprising gaboxadol or a pharmaceutically acceptable salt thereof wherein the second pharmaceutical composition provides an in vivo plasma profile comprising a mean AUC_{0-∞} of at least 20% less than the first pharmaceutical composition.

Embodiments described herein provide that a patient in need thereof is administered a pharmaceutical composition including gaboxadol or a pharmaceutically acceptable salt thereof. Gaboxadol or pharmaceutically acceptable salt thereof may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, sulfuric, sulfamic, phosphoric or nitric acid addition salts may be used.

In embodiments, gaboxadol is provided as gaboxadol monohydrate. One skilled in the art will readily understand that the amounts of active ingredient in a pharmaceutical composition will depend on the form of gaboxadol provided. For example, pharmaceutical compositions of including 5.0, 10.0, or 15.0 mg gaboxadol correspond to 5.6, 11.3, or 16.9 mg gaboxadol monohydrate.

In embodiments, gaboxadol is crystalline, such as the crystalline hydrochloric acid salt, the crystalline hydrobromic acid salt, or the crystalline zwitter ion monohydrate. In embodiments, gaboxadol is provided as a crystalline monohydrate.

In embodiments, allopregnanolone or pharmaceutically acceptable salt thereof may be provided as an acid addition salt, e.g., hydrochloric, hydrobromic, sulfuric, sulfamic, phosphoric or nitric acid, e.g., a sulfate. In embodiments allopregnanolone ma be a sulfate sodium salt or a tertraethylammonium salt.

Deuteration of pharmaceuticals to improve pharmacokinetics (PK), pharmacodynamics (PD), and toxicity profiles, has been demonstrated previously with some classes of drugs. Accordingly the use of deuterium enriched gaboxadol, allopregnanolone or ganaxolone is contemplated and within the scope of the methods and compositions described herein. Deuterium can be incorporated in any position in replace of hydrogen synthetically, according to the synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art to provide deuterium enriched gaboxadol, allopregnanolone or ganaxolone. See Journal of Labeled Compounds and Radiopharmaceuticals 19(5) 689-702 (1982).

Deuterium enriched gaboxadol, allopregnanolone or ganaxolone may be described by the percentage of incorporation of deuterium at a given position in the respective molecules in the place of hydrogen. For example, deuterium enrichment of 1% at a given position means that 1% of molecules in a given sample contain deuterium at that specified position. The deuterium enrichment can be determined using conventional analytical methods, such as mass spectrometry and nuclear magnetic resonance spectroscopy. In embodiments deuterium enriched gaboxadol means that the specified position is enriched with deuterium above the naturally occurring distribution (i.e., above about.0156%). In embodiments deuterium enrichment is no less than about 1%, no less than about 5%, no less than about 10%, no less than about 20%, no less than about 50%, no less than about 70%, no less than about 80%, no less than about 90%, or no less than about 98% of deuterium at a specified position.

As mentioned above, in embodiments, methods of treating cervical dystonia, multiple system atrophy, myoclonus, progressive supranuclear palsy, restless legs syndrome, Wilson's disease, ataxia, chorea, or dystonia include administering to a patient in need thereof a pharmaceutical composition including about 0.05 mg to about 30 mg gaboxadol or a pharmaceutically acceptable salt thereof.

For example, dosages may include amounts of gaboxadol or pharmaceutically acceptable salt thereof in the range of about, e.g., 0.05 mg to 50 mg, 1 mg to 30 mg, 1 mg to 20 mg, 1 mg to 15 mg, 0.01 mg to 10 mg, 0.1 mg to 15 mg, 0.1 mg to 30 mg, 0.15 mg to 12.5 mg, or 0.2 mg to 10 mg, with doses of 0.05 mg, 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, and 30 mg being specific examples of doses.

In embodiments, the pharmaceutical compositions include 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, 3 mg to 15 mg gaboxadol or a pharmaceutically acceptable salt thereof.

In embodiments, the pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, the pharmaceutical compositions include 0.1 mg, 0.25 mg, 0.5 mg, 1 mg, 2.5 mg, 3 mg, 4 mg, 5 mg, 7 mg, 7.5 mg, 8 mg, 8.5 mg, 9 mg, 9.5 mg, 10 mg, 10.5 mg, 11 mg, 12 mg, 12.5 mg, 13 mg, 14 mg, 15 mg, 16 mg, 17 mg, 17.5 mg, 18 mg, 19 mg, 20 mg, 21 mg, 22 mg, 23 mg, 24 mg, 25 mg, 26 mg, 27 mg, 28 mg, 29 mg, or 30 mg gaboxadol or a pharmaceutically acceptable salt thereof or amounts that are multiples of such doses. In embodiments, the pharmaceutical compositions include 2.5 mg, 5 mg, 7.5 mg, 10 mg, 15 mg, or 20 mg gaboxadol or a pharmaceutically acceptable salt thereof.

In embodiments, dosages of gaboxadol or a pharmaceutically acceptable salt thereof are administered once daily, twice daily, three times daily or four times daily to a patient in need thereof. The methods and compositions described herein may provide reduced dosing frequency and reduced adverse events and/or increased efficacy. In embodiments, the dosage is about, e.g., 0.05-30 mg/day, 0.1-20 mg/day, or 0.2-15 mg/day, or 0.5-10 mg/day, or 0.75-5 mg/day, for example 0.1 mg/day, 0.2 mg/day, 0.5 mg/day, 0.75 mg/day, 1 mg/day, 1.5 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 21 mg/day, 22 mg/day, 23 mg/day, 24 mg/day, 25 mg/day, 26 mg/day, 27 mg/day, 28 mg/day, 29 mg/day, or 30 mg/day. In embodiments, gaboxadol or a pharmaceutically acceptable salt thereof, or a derivative or analogue thereof, is administered at doses of 0.2 mg to 1 mg in infants or 1-20 mg in adults, once daily.

In embodiments, the total amount of gaboxadol or pharmaceutically acceptable salt thereof and/or gaboxadol administered to a subject in a 24-hour period is 1 mg to 50 mg. In embodiments, the total amount of gaboxadol or pharmaceutically acceptable salt thereof and/or gaboxadol administered to a subject in a 24-hour period is 1 mg to 20 mg. In embodiments, the total amount of gaboxadol or pharmaceutically acceptable salt thereof and/or gaboxadol administered to a subject in a 24-hour period is 5 mg, 10 mg, 15 mg or 20 mg. In embodiments, the total amount of gaboxadol or a pharmaceutically acceptable salt thereof administered to a subject in a 24-hour period is 1 mg to 50 mg. In embodiments, the subject may be started at a low dose and the dosage is escalated. In this manner, it can be determined if the drug is well tolerated in the subject. Dosages can be lower for children than for adults. In embodiments, a dose of gaboxadol for children can be 0.1 mg/kg to 1 mg/kg. In embodiments, gaboxadol or a pharmaceutically acceptable salt thereof is administered parenterally.

In embodiments, a method of treating cervical dystonia, multiple system atrophy, myoclonus, progressive supranuclear palsy, restless legs syndrome, Wilson's disease, ataxia, chorea, or dystonia includes administering allopregnanolone or a pharmaceutically acceptable salt thereof to a patient in need thereof. Allopregnanolone or a pharmaceutically acceptable salt thereof can be administered in doses ranging, e.g., from 0.01 mg/kg to 20 mg/kg, 0.02 mg/kg to 19 mg/kg, 0.03 mg/kg to 18 mg/kg, 0.04 mg/kg to 17 mg/kg, 0.05 mg/kg to 16 mg/kg, 0.06 mg/kg to 15 mg/kg, 0.07 mg/kg to 14 mg/kg, 0.08 mg/kg to 14 mg/kg, 0.09 mg/kg to 13 mg/kg, 0.1 mg/kg to 12 mg/kg, 0.2 mg/kg to 11 mg/kg, 0.3 mg/kg to 10 mg/kg, 0.4 mg/kg to 9 mg/kg, 0.5 mg/kg to 8 mg/kg, 0.6 mg/kg to 7 mg/kg, 0.7 mg/kg to 6 mg/kg, 0.8 mg/kg to 5 mg/kg, 0.9 mg/kg to 4 mg/kg, or 1 mg/kg to 3 mg/kg. In embodiments, allopregnanolone doses can be, e.g., 0.01 mg, 0.05 mg, 0.75 mg, 0.1 mg, 0.25 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, or 20 mg.

Allopregnanolone or a pharmaceutically acceptable salt thereof can be administered, e.g., once daily, twice daily, three times daily, or four times daily. In embodiments, allopregnanolone or a pharmaceutically acceptable salt thereof can be administered once weekly. In embodiments, allopregnanolone or a pharmaceutically acceptable salt thereof can be administered parenterally. In embodiments, allopregnanolone or a pharmaceutically acceptable salt thereof can be administered parenterally in escalating doses.

In embodiments, a method of treating an cervical dystonia, multiple system atrophy, myoclonus, progressive supranuclear palsy, restless legs syndrome, Wilson's disease, ataxia, chorea, or dystonia includes administering ganaxolone or a pharmaceutically acceptable salt thereof to a patient in need thereof. Ganaxolone or a pharmaceutically acceptable salt thereof can be administered in doses ranging from 10 mg/kg to 40 mg/kg, e.g., 11 mg/kg to 39 mg/kg, 12 mg/kg to 38 mg/kg, 13 mg/kg to 37 mg/kg, 14 mg/kg to 36 mg/kg, 15 mg/kg to 35 mg/kg, 16 mg/kg to 34 mg/kg, 17 mg/kg to 33 mg/kg, 18 mg/kg to 32 mg/kg, 19 mg/kg to 31 mg/kg, 20 mg/kg to 30 mg/kg, 21 mg/kg to 29 mg/kg, 22 mg/kg to 28 mg/kg, 23 mg/kg to 27 mg/kg, or 24 mg/kg to 26 mg/kg. In embodiments, ganaxolone doses can be, e.g., 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, or 2000 mg.

Ganaxolone or a pharmaceutically acceptable salt thereof can be administered, e.g., once daily, twice daily, three times daily, or four times daily. In embodiments, ganaxolone or a pharmaceutically acceptable salt thereof can be administered once weekly. In embodiments, ganaxolone or a pharmaceutically acceptable salt thereof can be administered parenterally. In embodiments, ganaxolone or a pharmaceutically acceptable salt thereof can be administered parenterally in escalating doses.

In embodiments, methods of treating cervical dystonia, multiple system atrophy, myoclonus, progressive supranuclear palsy, restless legs syndrome, Wilson's disease, ataxia, chorea, or dystonia by administering to a subject in need thereof an effective amount of gaboxadol or a pharmaceutically acceptable salt thereof, either alone or in combination with allopregnanolone or a pharmaceutically acceptable salt, derivative or analogue are provided. In embodiments, methods of treating cervical dystonia, multiple system atrophy, myoclonus, progressive supranuclear palsy, restless legs syndrome, Wilson's disease, ataxia, chorea, or dystonia by administering to a subject in need thereof an effective amount of gaboxadol or a pharmaceutically acceptable salt thereof, either alone or in combination with ganaxolone or a pharmaceutically acceptable salt, derivative or analogue are provided. In embodiments, methods of treating cervical dystonia, multiple system atrophy, myoclonus, progressive supranuclear palsy, restless legs syndrome, Wilson's disease, ataxia, chorea, or dystonia by administering to a subject in need thereof an effective amount of allopregnanolone or a pharmaceutically acceptable salt, derivative or analogue, or combination thereof, either alone or in combination with, gaboxadol or a pharmaceutically acceptable salt thereof, are provided. In embodiments, methods of treating cervical dystonia, multiple system atrophy, myoclonus, progressive supranuclear palsy, restless legs syndrome, Wilson's disease, ataxia, chorea, or dystonia by administering to a subject in need thereof an effective amount of ganaxolone or a pharmaceutically acceptable salt, derivative or analogue, or combination thereof, either alone or in combination with, gaboxadol or a pharmaceutically acceptable salt thereof, are provided.

An effective amount or therapeutically effective amount can be a dosage sufficient to treat, inhibit, or alleviate one or more respective symptoms of cervical dystonia, multiple system atrophy, myoclonus, progressive supranuclear palsy, restless legs syndrome, Wilson's disease, ataxia, chorea, or dystonia such as reducing the frequency or severity of increased movement, reducing the frequency or severity of decreased or slow movement, and/or reducing the frequency or severity of spastic movement. Other symptoms include long-lasting contractions (spasms) or intermittent contractions of the neck muscles (as in cervical dystonia), ataxia or parkinsonism (as in multiple system atrophy), lightning-quick jerks of a muscle or a group of muscles (as in myoclonus), problems with walking, balance and eye movements (as in progressive supranuclear palsy), unpleasant, abnormal feelings in the legs while relaxing or lying down (as in restless legs syndrome), uncoordinated or clumsy balance, speech or limb movements (ataxia), repetitive, brief, irregular, somewhat rapid, involuntary movements that typically involve the face, mouth, trunk and limbs (chorea), and sustained involuntary muscle contractions with twisting, repetitive movements (dystonia).

An effective amount or therapeutically effective amount can also provide a desired pharmacologic and/or physiologic effect, for example, reducing, inhibiting, or reversing one or more of the underlying pathophysiological mechanisms underlying the neurological dysfunction of the movement disorder. The precise dosage will vary according to a variety of factors such as subject-dependent variables (e.g., age, immune system health, clinical symptoms etc.). In embodiments, a subject may be started at a low dose and the dosage is escalated. In this manner, it can be determined if the drug is well tolerated in the subject. Dosages can be lower for children than for adults.

In embodiments, the methods described herein are effective to reduce, delay, or prevent one or more other clinical symptoms of cervical dystonia, multiple system atrophy, myocl onus, progressive supranuclear palsy, restless legs syndrome, Wilson's disease, ataxia, chorea, or dystonia described above. For example, the effect of a gaboxadol or a pharmaceutically acceptable salt thereof, and/or allopregnanolone or a pharmaceutically acceptable salt, derivative or analogue thereof, and/or ganaxolone or a pharmaceutically acceptable salt, derivative or analogue thereof, on a particular symptom, pharmacologic, or physiologic indicator can be compared to an untreated subject, or the condition of the subject prior to treatment. In embodiments, the symptom, pharmacologic, and/or physiologic indicator is measured in a subject prior to treatment, and again one or more times after treatment is initiated. In embodiments, the control is a reference level, or average determined based on measuring the symptom, pharmacologic, or physiologic indicator in one or more subjects that do not have the disease or condition to be treated (e.g., healthy subjects). In embodiments, the effect of the treatment is compared to a conventional treatment that is known the art.

In embodiments, compositions and methods of treatment are provided with low dosages of gaboxadol, and/or allopregnanolone or a pharmaceutically acceptable salt, derivative or analogue thereof, and/or ganaxolone or a pharmaceutically acceptable salt, derivative or analogue thereof such that the patient is provided one or more beneficial effects related to the movement disorders described above. Provided herein are dosing regimens that allow effective treatment of a movement disorder with potentially limited or substantially few negative side effects, e.g., convulsions and/or sleep disruption. Accordingly, the methods described herein may provide treatment of a movement disorder that may be considered surprising and unexpected. For example, methods are provided herein of treating movement disorders in a patient in need thereof which may not cause sleep disruption. In embodiments, methods described herein may provide effective treatment of a movement disorder without interrupting Slow Wave Sleep. In embodiments, methods of treating a movement disorder without causing insomnia or trouble falling asleep are provided.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides improvement of at least one cervical dystonia, multiple system atrophy, myoclonus, progressive supranuclear palsy, restless legs syndrome, Wilson's disease, ataxia, chorea, or dystonia symptom for more than 4 hours after administration of the pharmaceutical composition to the patient. In embodiments, the improvement of at least one cervical dystonia, multiple system atrophy, myoclonus, progressive supranuclear palsy, restless legs syndrome, Wilson's disease, ataxia, chorea, or dystonia symptom for more than 6 hours after administration of the pharmaceutical composition to the patient is provided in accordance with the present disclosure. In embodiments, improvement of at least one cervical dystonia, multiple system atrophy, myoclonus, progressive supranuclear palsy, restless legs syndrome, Wilson's disease, ataxia, chorea, or dystonia symptom for more than, e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one cervical dystonia, multiple system atrophy, myoclonus, progressive supranuclear palsy, restless legs syndrome, Wilson's disease, ataxia, chorea, or dystonia symptom for at least e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one cervical dystonia, multiple system atrophy, myoclonus, progressive supranuclear palsy, restless legs syndrome, Wilson's disease, ataxia, chorea, or dystonia symptom for 12 hours after administration of the pharmaceutical composition to the patient is provided in accordance with the present disclosure.

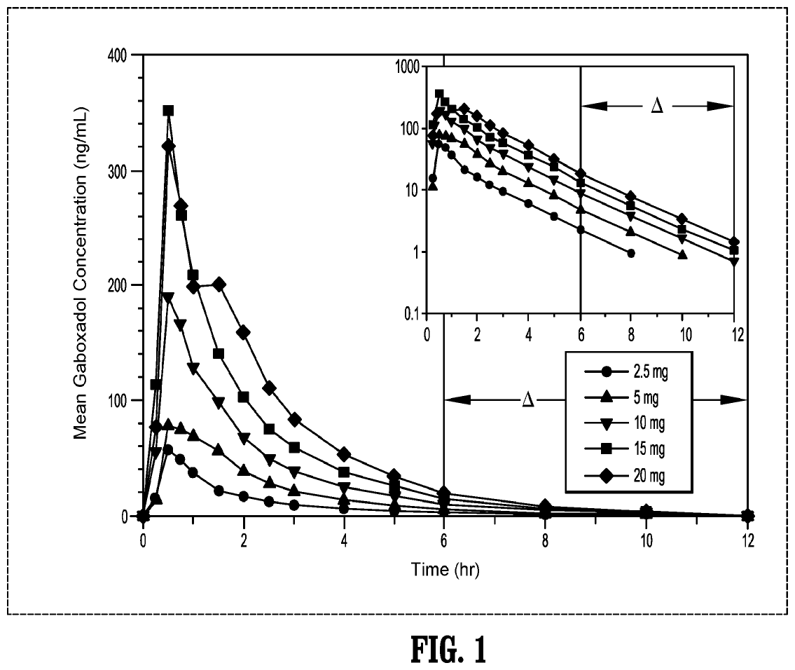

FIG. 1 shows the arithmetic mean plasma concentration-time profiles of gaboxadol following single oral doses (2.5, 5, 10, 15, and 20 mg)(see, Example 1, below) with horizontal lines Δ indicating the change between 6 and 12 hours. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 50% and the method provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 55% and the method provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 60% and the method provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 65% and the method provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 70% and the method provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof about 0.05 mg to about 30 mg gaboxadol or a pharmaceutically acceptable salt thereof which provides an in vivo plasma profile, wherein the in vivo plasma profile of the patient 6 hours after administration of the gaboxadol or pharmaceutically acceptable salt thereof is reduced by more than 75% and the method provides improvement in the patient for more than 6, 8, 10, 12, 14, 16, 18, 20, 22 or 24 hours after administration.

In embodiments, provided herein are methods of treating cervical dystonia, multiple system atrophy, myoclonus, progressive supranuclear palsy, restless legs syndrome, Wilson's disease, ataxia, chorea, or dystonia wherein the amount of gaboxadol, allopregnanolone, or ganaxolone, or a pharmaceutical salt of any of the foregoing, within the patient about 4 hours after administration of the pharmaceutical composition is less than about 75% of the administered dose. In embodiments, provided herein are methods wherein the amount of gaboxadol, allopregnanolone, or ganaxolone, or a pharmaceutical salt of any of the foregoing, within the patient about, e.g., 6 hours, 8 hours, 10 hours, 12 hours, 15 hours, or 20 hours after administration of the pharmaceutical composition is less than about 75%.

In embodiments, provided herein are methods of treating cervical dystonia, multiple system atrophy, myoclonus, progressive supranuclear palsy, restless legs syndrome, Wilson's disease, ataxia, chorea, or dystonia wherein the amount of gaboxadol, allopregnanolone, or ganaxolone, or a pharmaceutical salt of any of the foregoing, within the patient about 4 hours after administration of the pharmaceutical composition is less than about 80% of the administered dose. In embodiments, provided herein are methods wherein the amount of gaboxadol, allopregnanolone, or ganaxolone, or a pharmaceutical salt of any of the foregoing, within the patient about, e.g., 6 hours, 8 hours, 10 hours, 12 hours, 15 hours, or 20 hours after administration of the pharmaceutical composition is less than about 80% of the administered dose.

In embodiments, provided herein are methods of treating cervical dystonia, multiple system atrophy, myoclonus, progressive supranuclear palsy, restless legs syndrome, Wilson's disease, ataxia, chorea, or dystonia wherein the amount of gaboxadol, allopregnanolone, or ganaxolone, or a pharmaceutical salt of any of the foregoing, within the patient about 4 hours after administration of the pharmaceutical composition is between about 65% to about 85% of the administered dose. In embodiments, the amount of gaboxadol, allopregnanolone, or ganaxolone, or a pharmaceutical salt of any of the foregoing, within the patient after about, e.g., 6 hours, 8 hours, 10 hours, 12 hours, 15 hours, or 20 hours after administration of the pharmaceutical composition is between about 65% to about 85% of the administered dose.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides an in vivo plasma concentration 6 hours after administration which is less than 75% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides an in vivo plasma concentration 6 hours after administration which is less than 80% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides an in vivo plasma concentration 6 hours after administration which is less than 85% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides an in vivo plasma concentration 6 hours after administration which is less than 90% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides an in vivo plasma concentration 6 hours after administration which is less than 95% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol, allopregnanolone, or ganaxolone, or a pharmaceutical salt of any of the foregoing, wherein the composition provides an in vivo plasma concentration 6 hours after administration which is less than 100% of the administered dose and provides improvement in the patient for more than 6, 8, 10, 12, 14, 16, 18, 20, 22 or 24 hours after administration.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a Cₘₐₓ less than about 500 ng/ml. In embodiments, the composition provides improvement for more than 6 hours after administration to the patient.

In embodiments, the composition provides an in vivo plasma profile having a Cₘₐₓ less than about, e.g., 450 ng/ml, 400 ng/ml 350 ng/ml, or 300 ng/ml and wherein the composition provides improvement in one or more symptoms of cervical dystonia, multiple system atrophy, myoclonus, progressive supranuclear palsy, restless legs syndrome, Wilson's disease, ataxia, chorea, or dystonia a day after administration. In embodiments, the composition provides an in vivo plasma profile having a Cₘₐₓ less than about, e.g., 250 ng/ml, 200 ng/ml 150 ng/ml, or 100 ng/ml and wherein the composition provides improvement in one or more symptoms of cervical dystonia, multiple system atrophy, myoclonus, progressive supranuclear palsy, restless legs syndrome, Wilson's disease, ataxia, chorea, or dystonia a day after administration.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC_{0-∞}of less than about 900 ng·hr/ml. In embodiments, the composition provides improvement in one or more symptoms of cervical dystonia, multiple system atrophy, myoclonus, progressive supranuclear palsy, restless legs syndrome, Wilson's disease, ataxia, chorea, or dystonia a day after administration. In embodiments, the compositions provide an in vivo plasma profile having a AUC_{0-∞} of less than about, e.g., 850 ng·hr/ml, 800 ng·hr/ml, 750 ng·hr/ml, or 700 ng·hr/ml and wherein the composition provides improvement in one or more symptoms of cervical dystonia, multiple system atrophy, myoclonus, progressive supranuclear palsy, restless legs syndrome, Wilson's disease, ataxia, chorea, or dystonia a day after administration. In embodiments, the composition provides improvement in one or more cervical dystonia, multiple system atrophy, myoclonus, progressive supranuclear palsy, restless legs syndrome, Wilson's disease, ataxia, chorea, or dystonia symptoms for more than 6 hours after administration.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC_{0-∞}of less than about, e.g., 650 ng·hr/ml, 600 ng·hr/ml, 550 ng·hr/ml, 500 ng·hr/ml, or 450 ng·hr/ml. In embodiments, the composition provides an in vivo plasma profile having a AUC_{0-∞} of less than about, e.g., 400 ng·hr/ml, 350 ng·hr/ml, 300 ng·hr/ml, 250 ng·hr/ml, or 200 ng·hr/ml. In embodiments, the composition provides an in vivo plasma profile having a AUC_{0-∞} of less than about, e.g., 150 ng·hr/ml, 100 ng·hr/ml, 75 ng·hr/ml, or 50 ng·hr/ml. In embodiments, the composition provides improvement symptoms of cervical dystonia, multiple system atrophy, myoclonus, progressive supranuclear palsy, restless legs syndrome, Wilson's disease, ataxia, chorea, or dystonia for more than, e.g., 4 hours, 6 hours, 8 hours, 10 hours, or 12 hours, after administration of the composition to the patient.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof an amount of gaboxadol or a pharmaceutically acceptable salt thereof which provides an in vivo plasma profile having a AUC₆₋₁₂ which is less than 75% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof an amount of gaboxadol or a pharmaceutically acceptable salt thereof which provides an in vivo plasma profile having a AUC₆₋₁₂ which is less than 80% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof an amount of gaboxadol or a pharmaceutically acceptable salt thereof which provides an in vivo plasma profile having a AUC₆₋₁₂ which is less than 85% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof an amount of gaboxadol or a pharmaceutically acceptable salt thereof which provides an in vivo plasma profile having a AUC₆₋₁₂ which is less than 90% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof an amount of gaboxadol or a pharmaceutically acceptable salt thereof which provides an in vivo plasma profile having a AUC₆₋₁₂ which is less than 95% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof an amount of gaboxadol or a pharmaceutically acceptable salt thereof which provides an in vivo plasma profile having a AUC₆₋₁₂ which is less than 100% of the Cₘₐₓ and provides improvement in the patient for more than 6, 8, 10, 12, 14, 16, 18, 20, 22 or 24 hours after administration.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 75% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 80% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 85% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 90% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 95% of the Cₘₐₓ and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 100% of the Cₘₐₓ and provides improvement in the patient for more than 6, 8, 10, 12, 14, 16, 18, 20, 22 or 24 hours after administration.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 75% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 80% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 85% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 90% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 95% of the administered dose and provides improvement in the patient for more than 6, 8, 10, 12, 14, 16, 18, 20, 22 or 24 hours after administration. In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a pharmaceutical composition including gaboxadol or a pharmaceutically acceptable salt thereof wherein the composition provides an in vivo plasma profile having a AUC₆₋₁₂ which is less than 100% of the administered dose and provides improvement in the patient for more than 6, 8, 10, 12, 14, 16, 18, 20, 22 or 24 hours after administration.

In embodiments, provided herein are methods of treating cervical dystonia, multiple system atrophy, myoclonus, progressive supranuclear palsy, restless legs syndrome, Wilson's disease, ataxia, chorea, or dystonia including administering to a patient in need thereof a first pharmaceutical composition including gaboxadol, allopregnanolone, or ganaxolone, or a pharmaceutical salt of any of the foregoing, and a second pharmaceutical composition including gaboxadol, allopregnanolone, or ganaxolone, or a pharmaceutical salt of any of the foregoing, wherein the second pharmaceutical composition provides an in vivo plasma profile having a mean AUC_{0-∞} of at least about 20% less than the first pharmaceutical composition.

In embodiments, pharmaceutical compositions herein may be provided in the form of tablets, capsules, suppositories, films, inhalants, solutions, suspensions or emulsions. In embodiments, pharmaceutical compositions herein are suitable for parenteral administration, including, e.g., intramuscularly (i.m.), intravenously (i.v.), subcutaneously (s.c.), intraperitoneally (i.p.), or intrathecally (i.t.). The parenteral compositions herein must be sterile for administration by injection, infusion or implantation into the body and may be packaged in either single-dose or multi-dose containers. The parenteral compositions may be contained in a bag, a glass vial, a plastic vial, or a bottle.

In embodiments, the pharmaceutical compositions described herein are administered once, twice, three times or four times daily, every other day, or once a week. In embodiments, a pharmaceutical composition described herein is provided to the patient in the evening. In embodiments, a pharmaceutical composition described herein is provided to the patient in the morning. In embodiments, a pharmaceutical composition described herein is provided to the patient in the afternoon. In embodiments, a pharmaceutical composition described herein is provided to the patient once in the evening and once in the morning. In embodiments, a pharmaceutical composition herein is provided as soon as possible after the occurrence of symptoms. In embodiments, a pharmaceutical composition herein is provided continuously, e.g., by infusion.

Pharmaceutical compositions herein may be provided with conventional release profiles or modified release profiles. Conventional (or unmodified) release oral dosage forms such as tablets or capsules typically release medications into the stomach or intestines as the tablet or capsule shell dissolves. The pattern of drug release from modified release (MR) dosage forms is deliberately changed from that of a conventional dosage form to achieve a desired therapeutic objective and/or better patient compliance. Types of MR drug products include immediate release, delayed release and extended release dosage forms. Orally disintegrating dosage forms (ODDFs) provide immediate release of medication. In embodiments, pharmaceutical compositions with different drug release profiles may be combined to create a two phase or three-phase release profile. For example, pharmaceutical compositions may be provided with an immediate release and an extended release profile. In embodiments, pharmaceutical compositions may be provided with an extended release and delayed release profile. Such composition may be provided as pulsatile formulations, multilayer tablets, or capsules containing tablets, beads, granules, etc. Enteric coated dosage forms are an example of delayed release dosage forms. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. In embodiments, when administered to an oral cavity, an ODDF herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds.

An orally disintegrating tablet (ODT) is a solid dosage form containing a medicinal substance or active ingredient which disintegrates rapidly, usually within a matter of seconds when placed upon the tongue. The disintegration time for ODTs generally ranges from several seconds to about a minute. ODTs are designed to disintegrate or dissolve rapidly on contact with saliva, thus eliminating the need to chew the tablet, swallow the intact tablet, or take the tablet with liquids. In embodiments, an ODT herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds, based upon, e.g., the United States Pharmacopeia (USP) disintegration test method set forth at section 701, Revision Bulletin Official Aug. 1, 2008.

Other ODDFs which may be used herein include rapidly dissolving films which are thin oral strips that release medication such as gaboxadol, allopregnanolone, or ganaxolone, or a pharmaceutical salt of any of the foregoing, quickly after administration to the oral cavity. The film is placed on a patient's tongue or any other mucosal surface and is instantly wet by saliva whereupon the film rapidly hydrates and dissolves to release the medication. Fastcaps are a rapidly disintegrating drug delivery system based on gelatin capsules. Freeze dried (lyophilized) wafers are rapidly disintegrating, thin matrixes that contain a medicinal agent. The wafer or film disintegrates rapidly in the oral cavity and releases drug which dissolves or disperses in the saliva. Those skilled in the art are familiar with various techniques utilized to manufacture ODDFs such as freeze drying, spray drying, phase transition processing, melt granulation, sublimation, mass extrusion, cotton candy processing, direct compression, etc.

When administered, ODDFs containing gaboxadol, allopregnanolone, or ganaxolone, or a pharmaceutical salt of any of the foregoing, either alone or with one or more additional drugs discussed herein (collective referred to herein as “drug”, “drugs”, “active agent”, or “active agents”), disintegrate rapidly to release the drug(s), which dissolves or disperses in the saliva. The drug may be absorbed in the oral cavity, e.g., sublingually, buccally, from the pharynx and esophagus or from other sections of gastrointestinal tract as the saliva travels down. In such cases, bioavailability can be significantly greater than that observed from conventional tablet dosage forms which travel to the stomach or intestines where drug can be released.

In embodiments, pharmaceutical compositions having modified release profiles provide pharmacokinetic properties which result in both rapid onset and sustained duration of action. Such pharmaceutical compositions include an immediate release aspect and an extended release aspect. Immediate release aspects are discussed above in connection with ODDFs. Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. In embodiments, modified release dosage forms herein are ERDFs that do not have an ODDF aspect. In embodiments, modified release dosage forms herein incorporate an ODDF aspect to provide immediate release of a loading dose and then an ERDF aspect that provides prolonged delivery to maintain drug levels in the blood within a desired therapeutic range for a desirable period of time in excess of the activity resulting from a single dose of the drug. In embodiments, the ODDF aspect releases the drug immediately and the ERDF aspect thereafter provides continuous release of drug for sustained action.

Suitable formulations which provide extended release profiles are well-known in the art. For example, coated slow release beads or granules (“beads” and “granules” are used interchangeably herein) in which, e.g., gaboxadol or a pharmaceutically acceptable salt thereof, alone or in combination with one or more drugs, is applied to beads, e.g., confectioners nonpareil beads, and then coated with conventional release retarding materials such as waxes, enteric coatings and the like. In embodiments, some beads incorporate one drug while other beads incorporate a different drug. In embodiments, beads can be formed in which one or more drugs are mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets. In embodiments, extended release profiles may be provided by multiple layer tablets, each layer having different release properties. Multilayer tableting machines allow incorporation into one tablet of two or more separate layers which may be made to release one or more drugs at different rates.

In embodiments, one or more drugs are incorporated into porous inert carriers that provide extended release profiles. In embodiments, the porous inert carriers incorporate channels or passages from which the drug diffuses into surrounding fluids. In embodiments, one or more drugs are incorporated into an ion-exchange resin to provide an extended release profile. Prolonged action results from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide an extended release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads. In embodiments, absorbable (e.g., glycolide) or non-absorbable polymers may be utilized to form ERDFs. Various ERDFs including those discussed above and others that can be utilizable herein are known to those with skill in the art.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as gaboxadol, allopregnanolone, or ganaxolone, or a pharmaceutical salt of any of the foregoing, at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, with respect to tablets or capsules, enteric-coated articles are examples of delayed release dosage forms. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

Delayed release dosage forms are known to those skilled in the art. For example, coated delayed release beads or granules (“beads” and “granules” are used interchangeably herein) in which, e.g., gaboxadol, allopregnanolone, or ganaxolone, or a pharmaceutical salt of any of the foregoing, and/or other drug is applied to beads, e.g., confectioners nonpareil beads, and then coated with conventional release delaying materials such as waxes, enteric coatings and the like. In embodiments, beads can be formed in which drug is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of drug can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, modified release pharmaceutical compositions herein include pulsatile release dosage formulations (PRDFs). Pulsatile drug release involves rapid release of defined or discrete amounts of a drug (or drugs) such as gaboxadol or a pharmaceutically acceptable salt thereof after a lag time following an initial release of drug. In embodiments, PRDFs can provide a single pulse. In embodiments, PRDFs can provide multiple pulses over time. Various PRDFs are known to those with skill in the art.

In embodiments, liquid pharmaceutical compositions for parenteral administration to a subject include an active substance, e.g., gaboxadol, allopregnanolone, or ganaxolone, or a pharmaceutical salt of any of the foregoing, at a concentration of about 0.005 μg/ml to about 500 μg/ml. In embodiments, the composition includes an active substance, e.g., gaboxadol, allopregnanolone, or ganaxolone, or a pharmaceutical salt of any of the foregoing, at a concentration of, e.g., about 0.005 μg/ml to about 250 μg/ml, about 0.005 μg/ml to about 200 μg/ml, about 0.005 μg/ml to about 150 μg/ml, about 0.005 μg/ml to about 100 μg/ml, or about 0.005 μg/ml to about 50 μg/ml.

In embodiments, the compositions includes an active substance, e.g., gaboxadol, allopregnanolone, or ganaxolone, or a pharmaceutical salt of any of the foregoing, at a concentration of, e.g., about 0.05 μg/ml to about 50 μg/ml, about 0.1 μg/ml to about 50 μg/ml, about 0.05 μg/ml to about 25 μg/ml, about 0.05 μg/ml to about 10 μg/ml, about 0.05 μg/ml to about 5 μg/ml, or about 0.05 μg/ml to about 1 μg/ml. In embodiments, the composition includes an active substance, e.g., gaboxadol, allopregnanolone, or ganaxolone, or a pharmaceutical salt of any of the foregoing, at a concentration of, e.g., about 0.05 μg/ml to about 15 μg/ml, about 0.5 μg/ml to about 10 μg/ml, about 0.5 μg/ml to about 7 μg/ml, about 1 μg/ml to about 10 μg/ml, about 5 μg/ml to about 10 μg/ml, or about 5 μg/ml to about 15 μg/ml. In embodiments, the pharmaceutical compositions for parenteral administration is formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

In embodiments, compositions for parenteral administration include about 0.05 mg to about 100 mg active substance, e.g., gaboxadol, allopregnanolone, or ganaxolone, or a pharmaceutical salt of any of the foregoing. In embodiments, the pharmaceutical compositions include about, e.g., 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, 3 mg to 15 mg active substance, e.g., gaboxadol, allopregnanolone, or ganaxolone, or a pharmaceutical salt of any of the foregoing.

In embodiments, pharmaceutical compositions include about, e.g., 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg active substance, e.g., gaboxadol, allopregnanolone, or ganaxolone, or a pharmaceutical salt of any of the foregoing. In embodiments, the pharmaceutical compositions include about, e.g., 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, 20 mg active substance, e.g., gaboxadol, allopregnanolone, or ganaxolone, or a pharmaceutical salt of any of the foregoing, or amounts that are multiples of such doses. The compositions may be contained in a bag, a glass vial, a plastic vial, or a bottle.

In embodiments, pharmaceutical compositions for parenteral administration to a subject include an active substance, e.g., gaboxadol, allopregnanolone, or ganaxolone, or a pharmaceutical salt of any of the foregoing, at a concentration of about 0.005 mg/ml to about 500 mg/ml. In embodiments, the compositions include an active substance, e.g., gaboxadol, allopregnanolone, or ganaxolone, or a pharmaceutical salt of any of the foregoing, at a concentration of, e.g., about 0.05 mg/ml to about 50 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the composition includes an active substance, e.g., gaboxadol, allopregnanolone, or ganaxolone, or a pharmaceutical salt of any of the foregoing, at a concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml. In embodiments, pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are packages and stored in a bag, a glass vial, a plastic vial, or a bottle.

In embodiments, pharmaceutical compositions herein include an active substance, e.g., gaboxadol, allopregnanolone, or ganaxolone, or a pharmaceutical salt of any of the foregoing, wherein the active substance is present at a molarity less than about 1.0 M. In embodiments, the active substance, e.g., gaboxadol, allopregnanolone, or ganaxolone, or a pharmaceutical salt of any of the foregoing, is present at a molarity greater than, e.g., about 0.0001 M about 0.001 M, about 0.01 M, about 0.1 M, about 0.2 M, greater than about 0.5, greater than about 1.0 M, greater than about 1.2 M, greater than about 1.5 M, greater than about 1.75 M, greater than about 2.0 M, or greater than about 2.5 M. In embodiments, the active substance, e.g., gaboxadol, allopregnanolone, or ganaxolone, or a pharmaceutical salt of any of the foregoing, is present at a molarity of between, e.g., about 0.00001 M to about 0.1 M, about 0.01 to about 0.1 M, about 0.1 M to about 1.0 M, about 1.0 M to about 5.0 M, or about 5.0 M to about 10.0 M. In embodiments, the active substance, e.g., gaboxadol, allopregnanolone, or ganaxolone, or a pharmaceutical salt of any of the foregoing, is present at a molarity of less than, e.g., about 0.01 M, about 0.1 M, about 1.0 M, about 5.0 M, or about 10.0 M.

In embodiments, the solubility of the active substance, e.g., gaboxadol, allopregnanolone, or ganaxolone, or a pharmaceutical salt of any of the foregoing, in the composition is greater than, e.g., about 10 mg/mL, about 15 mg/mL, about 20 mg/mL, about 25 mg/mL, about 30 mg/mL, about 40 mg/mL, about 50 mg/mL, about 75 mg/mL, about 100 mg/mL, about 150 mg/mL, when measured, for example, in water at 25° C.

In embodiments, the solubility of the active substance, e.g., gaboxadol, allopregnanolone, or ganaxolone, or a pharmaceutical salt of any of the foregoing, in the composition is between, e.g., about 1 mg/mL to about 50 mg/mL, about 5 mg/mL to about 50 mg/mL, about 10 mg/mL to about 50 mg/mL, about 20 mg/mL to about 50 mg/ml, from about 20 mg/mL to about 30 mg/mL or from about 10 mg/mL to about 45 mg/mL, when measured, for example, in water at 25 C.

In embodiments, a pharmaceutical composition for parenteral administration is provided wherein the pharmaceutical composition is stable for at least six months. In embodiments, the pharmaceutical compositions herein exhibit no more than about 5% decrease in active substance, e.g., gaboxadol, allopregnanolone, or ganaxolone, or a pharmaceutical salt of any of the foregoing, e.g., 3 months or 6 months. In embodiments, the amount of gaboxadol or pharmaceutically acceptable salt thereof degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions are provided that are stable, soluble, local site compatible and/or ready-to-use. In embodiments, the pharmaceutical compositions herein are ready-to-use for direct administration to a patient in need thereof.

The parenteral compositions provided herein may include one or more excipients, solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of gaboxadol, allopregnanolone, or ganaxolone, or a pharmaceutical salt of any of the foregoing, used in the composition. Thus, parenteral compositions are provided wherein there is no incompatibility between any of the components of the dosage form.

In embodiments, parenteral compositions of gaboxadol, allopregnanolone, or ganaxolone, or a pharmaceutical salt of any of the foregoing, include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, pharmaceutical compositions include gaboxadol, allopregnanolone, or ganaxolone, or a pharmaceutical salt of any of the foregoing, and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments pharmaceutical compositions are provided including gaboxadol, allopregnanolone, or ganaxolone, or a pharmaceutical salt of any of the foregoing, and an excipient wherein the excipient is present in a molar ratio of the excipient to gaboxadol or pharmaceutically acceptable salt of, e.g., about 0.01:1 to about 0.45:1, about 0.1:1 to about 0.15:1, about 0.01:1 to about 0.1:1, and about 0.001:1 to about 0.01:1 are provided. In embodiments, the excipient is present at a molar ratio of the excipient to gaboxadol, allopregnanolone, or ganaxolone, or a pharmaceutical salt of any of the foregoing, is about 0.0001:1 to about 0.1:1 or about 0.001:1 to about 0.001:1.

In embodiments, pharmaceutical compositions are provided including gaboxadol, allopregnanolone, or ganaxolone, or a pharmaceutical salt of any of the foregoing, and an excipient wherein the excipient includes a stabilizing amount of a buffering agent. The buffering agent may be used to maintain the pH of the pharmaceutical composition wherein the gaboxadol, allopregnanolone, or ganaxolone, or a pharmaceutical salt of any of the foregoing, remains soluble, stable, and/or physiologically compatible. For example, in embodiments, the parenteral compositions include a buffering agent wherein the composition remains stable without significant gaboxadol, allopregnanolone, or ganaxolone, or a pharmaceutical salt of any of the foregoing, degradation. In embodiments, the addition of a buffer is desired for controlling the pH to enhance stability without significantly catalyzing or degrading the gaboxadol, allopregnanolone, or ganaxolone, or a pharmaceutical salt of any of the foregoing, and/or causing pain to the patient upon infusion.

In embodiments, the buffering agent can be a citrate, phosphate, acetate, tartrate, carbonate, glutamate, lactate, succinate, bicarbonate buffer and combinations thereof. For example, sodium citrate, trisodium citrate anhydrous, trisodium citrate dihydrate, sodium citrate dehydrate, triethanolamine (TRIS), trisodium citrate pentahydrate dihydrate (i.e., trisodium citrate dehydrate), acetic acid, citric acid, glutamic acid, phosphoric acid, may be used as a buffering agent. In embodiments, the buffering agent may be an amino acid, alkali metal, or alkaline earth metal buffer. For example, the buffering agent may be sodium acetate or hydrogen phosphate.

In embodiments, parenteral compositions of an active substance, e.g., gaboxadol, allopregnanolone, or ganaxolone, or a pharmaceutical salt of any of the foregoing, are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

In embodiments, pharmaceutical compositions of an active substance, e.g., gaboxadol, allopregnanolone, or ganaxolone, or a pharmaceutical salt of any of the foregoing, and an excipient wherein the excipient includes a solubilizing agent. For example, solubilizing agents according to the invention may include, e.g., sodium hydroxide, L-lysine, L-arginine, sodium carbonate, potassium carbonate, sodium phosphate, and/or potassium phosphate. The amount of solubilizing agent in the composition will be sufficient such that the solution remains soluble at all concentrations, i.e., does not turn hazy and/or form precipitates.

In embodiments, provided herein are pharmaceutical compositions of an active substance, e.g., gaboxadol, allopregnanolone, or ganaxolone, or a pharmaceutical salt of any of the foregoing, and an excipient wherein the excipient includes a particulate formation inhibitor. A particulate formation inhibitor refers to a compound that has the desired property of inhibiting the formation of particles in parenteral compositions. Particulate formation inhibitors of the invention include ethylenediaminetetraacetic acid (EDTA) and salts thereof, for example, ethylenediaminetetraacetic acid, calcium disodium salt (preferably as the hydrate); ethylenediaminetetraacetic acid, diammonium salt (e.g., as the hydrate); ethylenediaminetetraacetic acid, dipotassium salt (e.g., as the dihydrate); ethylenediaminetetraacetic acid, disodium salt (e.g., as the dihydrate and, if desired, as the anhydrous form); ethylenediaminetetraacetic acid, tetrasodium salt (e.g., as the hydrate); ethylenediaminetetraacetic acid, tripotassium salt (e.g., as the dihydrate); ethylenediaminetetraacetic acid, trisodium salt (preferably as the hydrate) and ethylenediaminetetraacetic acid disodium salt, USP (e.g., as the dihydrate). In embodiments, pharmaceutical compositions described herein have an effective amount of a particulate formation inhibitor. In embodiments the excipients may include, e.g., an amino acid, urea, alcohol, ascorbic acid, phospholipids, proteins, such as serum albumin, collagen, and gelatin; salts such as EDTA or EGTA, and sodium chloride, liposomes, polyvinylpyrollidone, sugars, such as dextran, mannitol, sorbitol, and glycerol, propylene glycol and polyethylene glycol (e.g., PEG-4000, PEG-6000), glycerol, glycine, and/or lipids.

In embodiments, provided herein are pharmaceutical compositions of an active substance, e.g., gaboxadol, allopregnanolone, or ganaxolone, or a pharmaceutical salt of any of the foregoing, and an excipient wherein the excipient includes a solubilizing agent. For example, solubilizing agents may include, but are not limited to, acids, such as carboxylic acids, amino acids. In other examples, the solubilizing agents may be saturated carboxylic acids, unsaturated carboxylic acids, fatty acids, keto acids, aromatic carboxylic acids, dicarboxylic acids, tricarboxylic acids, α-hydroxy acids, amino acids, and combinations thereof.

In embodiments, provided herein are pharmaceutical compositions of an active substance, e.g., gaboxadol, allopregnanolone, or ganaxolone, or a pharmaceutical salt of any of the foregoing, and an excipient wherein the excipient includes a solubilizing agent such as formic acid, acetic acid, propionic acid, butyric acid, valeric acid, caproic acid, enanthic acid, caprylic acid, pelargonic acid, capric acid, lauric acid, stearic acid, acrylic acid, docosahexaenoic acid, eicosapentaenoic acid, pyruvic acid, benzoic acid, salicylic acid, aldaric acid, oxalic acid, malonic acid, malic acid, succinic acid, glutaric acid, adipic acid, citric acid, lactic acid, alanine, arginine, aspargine, aspartic acid, cysteine, glutamine, glycine, histidine, isoleucine, leucine, lysine, methionine, phenylalanine, praline, serine, threonine, tryptophan, tyrosine, valine, and combinations thereof.

In embodiments, the solubilizing agent is selected from acetic acid, salts thereof, and combinations thereof, (e.g., acetic acid/sodium acetate), citric acid, salts thereof and combinations thereof (e.g., citric acid/sodium citrate), DL arginine, L-arginine and histadine. In embodiments, the solubilizing agent is DL-arginine. In embodiments, the solubilizing agent is L-arginine. In embodiments, the solubilizing agent is acetic acid/sodium acetate. In embodiments, the solubilizing agent is citric acid/sodium citrate.

In embodiments, provided herein are pharmaceutical compositions of an active substance, e.g., gaboxadol, allopregnanolone, or ganaxolone, or a pharmaceutical salt of any of the foregoing, and an excipient wherein the excipient renders the composition isotonic. Isotonic pharmaceutical compositions herein may be achieved by adding an appropriate quantity of sodium chloride, glucose, laevulose, dextrose, mannitol, or postassium chloride, or calcium chloride, or calcium gluconoglucoheptonate, or mixtures thereof. For example, the excipients may include one or more tonicity agents, such as, e.g., sodium chloride, potassium chloride, glycerin, mannitol, and/or dextrose. Tonicity agents may be used to minimize tissue damage and irritation, reduce hemolysis of blood cells, and/or prevent electrolyte imbalance. For example, the parenteral compositions may be an aqueous solution including sodium chloride wherein the composition is isotonic. In embodiments, the isotonizing agent is sodium chloride. In embodiments, the concentration of the isotonizing agent is between about 0.01 and about 2.0 weight percent. In embodiments, the pharmaceutical compositions may include up to about 10% isotonizing agent. In embodiments the pharmaceutical compositions may include up to about, e.g., 0.25%, 0.5%, 1%, 2.5% isotonizing agent. In embodiments the amount of isotonizing agent in the pharmaceutical is between about, e.g., 0.01% to 1%, 0.1% to 1%, 0.25% to 1%, or 0.5% to 1%.

In embodiments, provided herein are pharmaceutical compositions of an active substance, e.g., gaboxadol, allopregnanolone, or ganaxolone, or a pharmaceutical salt of any of the foregoing, and an excipient wherein the excipient includes a free radical antagonist. In embodiments, the free radical antagonist is ascorbic acid, ascorbic acid derivatives, organic compounds having at least one thiol, alkyl polyhydroxylated, and cycloalkyl polyhydroxylated compounds, and combinations thereof.

In embodiments, provided herein are pharmaceutical compositions of an active substance, e.g., gaboxadol, allopregnanolone, or ganaxolone, or a pharmaceutical salt of any of the foregoing, and an excipient wherein the excipient includes a free radical scavenger selected from thiolyglycolic acid, thiolacetic acid, dithiothreitol, reduced glutathion, thiourea, α-thioglycerol, cystein, aceticystein, mercaptoethane sulfonic acid and combinations thereof.

In embodiments, provided herein are pharmaceutical of an active substance, e.g., gaboxadol, allopregnanolone, or ganaxolone, or a pharmaceutical salt of any of the foregoing, and an excipient wherein the excipient includes ribolflavin, dithiothreitol, sodium thiosulfate, thiourea, ascorbic acid, methylene blue, sodium metabisulfite, sodium bisulfite, propyl gallate acetylcysteine, phenol, acetone sodium bisulfate, ascorbic acid, ascorbic acid esters, butylhydroxyanisol (BHA), Butylhydroxytoluene (BHT), cysteine, nordihydroguiaretic acid (NDGA), monothioglycerol, sodium bisulfite, sodium metabisulfate, tocophenols, and/or glutathione.

In embodiments, provided herein are pharmaceutical compositions of an active substance, e.g., gaboxadol, allopregnanolone, or ganaxolone, or a pharmaceutical salt of any of the foregoing, and an excipient wherein the excipient includes a preservative. In embodiments, the preservative is selected from benzalkonium chloride, benzethonium chloride, benzyl alcohol, chlorobutanol, chlorocresol, metacresol, Phenol, phenylmercuric nitrate, phenylmercuric acetate, methyl p-hydroxybenzoate, propyl p-hydroxybenzoate, butyl p-hydroxybenzoate, and thimerosal. In other embodiments, the preservative is selected from the group consisting of phenol, meta-cresol, benzyl alcohol, parabens (e.g., methyl, propyl, butyl), benzalkonium chloride, chlorobutanol, thimerosal, phenylmercuric salts (e.g., acetate, borate, or nitrate), and combinations thereof.

In embodiments, the compositions herein can include a co-solvent. For example, in some instances the solubility of gaboxadol, allopregnanolone, or ganaxolone, or a pharmaceutical salt of any of the foregoing, may be well below the therapeutic dose and therefore a co-solvent system may be used. A co-solvent is a mixture of solvents that may be used to achieve sufficiently high solubility and may increase the stability. For example, co-solvents may be a water-miscible organic solvents, such as ethanol, propylene, glycol, Capmul PG, propylene glycol, glycerin, polyethylene glycol, sorbitol, dimethylacetamide, and/or dimethylsulfoxide (DMSO). In embodiments, the cosolvent may encompass up to about 75% of the pharmaceutical composition. In other embodiments the amount of cosolvent used include up to about, e.g., 1%, 5%, 10%, 15%, 25%, 40%, 50%, of the pharmaceutical composition.

The dosage forms may be prepared, for example, by mixing of gaboxadol, allopregnanolone, or ganaxolone, or a pharmaceutical salt of any of the foregoing, and one or more excipients (e.g., buffering agents, solubilizing agents, tonicity agents, antioxidants, chelating agents, antimicrobial agents and/or preservatives) in a blender under sterile conditions until a uniform blend is obtained. Pre-sterilized vials may then be filled with an appropriate amount of the sterile blend. The predetermined amount of sterile blend may then be mixed with a solvent, e.g., water, saline, about 5-10% sugar (e.g., glucose, dextrose) solution and combinations thereof prior to administration. In addition, the solution may be frozen and thawed prior to further processing.

The excipients may be used in solid or in solution form. When used in solid form, the excipients and a gaboxadol, allopregnanolone, or ganaxolone, or a pharmaceutical salt of any of the foregoing, may be mixed together as described above, and then solvent added prior to parenteral administration. When used in solution form, the gaboxadol, allopregnanolone, or ganaxolone, or a pharmaceutical salt of any of the foregoing, may be mixed with a solution of the excipient prior to parenteral administration.

Parenteral solutions including gaboxadol, allopregnanolone, or ganaxolone, or a pharmaceutical salt of any of the foregoing, may be prepared by mixing the required amount of gaboxadol, allopregnanolone, or ganaxolone, or a pharmaceutical salt of any of the foregoing, which may be purified prior to use in parenteral fluids such as D5W, distilled water, saline or PEG and adjusting the pH of this solution between 6.8-8. The process may be carried out at room temperature, or to increase concentration, the solution may be warmed appropriately. Other solvents such as PEG 400, 600, polypropylene glycol or other glycols can be used to enhance solubility. The resulting solutions after cooling to room temperature, may be sterilized by known means such as ultrafiltration using, e.g., 0.45 micron filter or ethylene oxide treatment or heating and may be packaged into ampules, vials or pre-filled syringes suitable for dispensing a sterile parenteral formulation.

When administered, the parenteral compositions herein provide a time of maximum plasma concentration (Tₘₐₓ) for gaboxadol in human patients of about 1 or more hours (e.g., about 1.5 or more hours). In embodiments, a Tₘₐₓ of gaboxadol in human patients ranging from between, e.g., about 1 to about 5 hours, about 1 to about 4 hours, about 1 to about 3 hours, about 1 to about 2 hours. In embodiments, a Tₘₐₓ for gaboxadol in human patients of more than about 1.5 is observed. In embodiments, a Tₘₐₓ for gaboxadol in human patients of less than about 3 hours is observed. The time of maximum plasma concentration is measured once infusion is complete.

In embodiments herein a dosage form includes from about 1 mg to about 500 mg gaboxadol, wherein parenteral administration (e.g., intramuscular, intravenous, subcutaneous, intraperitoneal, or intrathecal) of the dosage form provides an in vivo plasma profile for gaboxadol encompassing a mean AUC_{0-∞} of more than about 25 ng·hr/ml. In embodiments, single dose administration of the dosage form provides an in vivo plasma profile for gaboxadol encompassing a mean AUC_{0-∞} of more than about, e.g., 50 ng·hr/ml, 75 ng·hr/ml, 150 ng·hr/ml, 250 ng·hr/ml, 500 ng·hr/ml, 1000 ng·hr/ml, or 1500 ng·hr/ml.

In embodiments, the dosage form includes from about 1 mg to about 500 mg gaboxadol, wherein administration of the dosage form provides an in vivo plasma profile for gaboxadol encompassing a mean Cₘₐₓ of less than about 10000 ng/ml. In embodiments, single dose administration of the compositions provide an in vivo plasma profile for gaboxadol of a mean Cₘₐₓ of less than about, e.g., 5000 ng/ml, 2500 ng/ml, 1000 ng/ml, 500 ng/ml, 250 ng/ml, or 100 ng/ml.

In embodiments, pharmaceutical compositions for parenteral administration include gaboxadol or a pharmaceutically acceptable salt thereof wherein parenteral administration exhibits a pharmacokinetic profile of a Tₘₐₓ at about 1 to about 120 minutes after administration of the parenteral composition; followed by a plasma drug concentration of at least 50% Cₘₐₓ for a duration of about 90 to about 360 minutes. In embodiments, parenteral administration of gaboxadol is followed by a plasma drug concentration of at least 50% Cₘₐₓ for a duration of, e.g., about 10 to about 60 minutes, about 15 to about 90 minutes, about 30 to about 120 minutes, about 60 to about 180 minutes, about 90 to about 180 minutes.

In embodiments, stable pharmaceutical compositions are provided in unit dosage form in a vial or ampoule suitable for parenteral administration having a therapeutically effective amount of gaboxadol, allopregnanolone, or ganaxolone, or a pharmaceutical salt of any of the foregoing, dissolved in sterile water to form a solution wherein the composition is substantially free of any excipient, organic solvent, buffer, acid, base, salt other than gaboxadol, allopregnanolone, or ganaxolone, or a pharmaceutical salt of any of the foregoing. In embodiments, the pharmaceutical composition remains sufficiently soluble and is capable of direct administration. In embodiments, the pharmaceutical composition is capable of storage in the absence of an inert atmosphere for at least 6 months.

In embodiments, provided herein are stable pharmaceutical compositions in unit dosage form in a vial or ampoule suitable for parenteral administration having a therapeutically effective amount of gaboxadol, allopregnanolone, or ganaxolone, or a pharmaceutical salt of any of the foregoing, dissolved in sterile water to form a solution wherein the composition is free of any excipient, organic solvent, buffer, acid, base, salt other than gaboxadol, allopregnanolone, or ganaxolone, or a pharmaceutical salt of any of the foregoing. In embodiments, the pharmaceutical composition remains sufficiently soluble and is capable of direct administration. In embodiments, the pharmaceutical composition is capable of storage in the absence of an inert atmosphere for at least 6 months.

In embodiments, stable pharmaceutical compositions suitable for parenteral administration include a gaboxadol, allopregnanolone, or ganaxolone, or a pharmaceutical salt of any of the foregoing, in an aqueous solution having an osmolarity between 225 and 350 mOsm/kg and at a pH in the range between 7.0 and 8.0. In embodiments, the aqueous solution has an osmolarity between 270 and 310. In embodiments, the aqueous solution has a pH in the range between 7.2 and 7.8.

In embodiments, provided herein are methods of treating an cervical dystonia, multiple system atrophy, myoclonus, progressive supranuclear palsy, restless legs syndrome, Wilson's disease, ataxia, chorea, or dystonia including administering to a patient in need thereof a first pharmaceutical dosage including a sub-therapeutic dosage of gaboxadol, allopregnanolone, or ganaxolone, or a pharmaceutical salt of any of the foregoing, wherein the composition provides improvement for more than 6 hours after administration.

In embodiments, provided herein are methods of treating an cervical dystonia, multiple system atrophy, myoclonus, progressive supranuclear palsy, restless legs syndrome, Wilson's disease, ataxia, chorea, or dystonia including administering to a patient in need thereof a first pharmaceutical dosage including a sub therapeutic dosage of gaboxadol or a pharmaceutically acceptable salt thereof wherein the composition provides improvement for more than 6 hours after administration.

In embodiments, provided herein are methods of treating an cervical dystonia, multiple system atrophy, myoclonus, progressive supranuclear palsy, restless legs syndrome, Wilson's disease, ataxia, chorea, or dystonia including administering to a patient in need thereof a first pharmaceutical composition including gaboxadol, allopregnanolone, or ganaxolone, or a pharmaceutical salt of any of the foregoing, and a second pharmaceutical composition including gaboxadol or a pharmaceutically acceptable salt thereof wherein the second pharmaceutical composition provides an in vivo plasma profile having a mean AUC_{0-∞} of less than about 900 ng·hr/ml. In embodiments, the second pharmaceutical composition provides an in vivo plasma profile having a AUC_{0-∞} of less than about, e.g., 800 ng·hr/ml, 750 ng·hr/ml, 700 ng·hr/ml, 650 ng·hr/ml, or 600 ng·hr/ml. In embodiments, the second pharmaceutical composition provides an in vivo plasma profile having a AUC_{0-∞} of less than about, e.g., 550 ng·hr/ml, 500 ng·hr/ml, 450 ng·hr/ml, 400 ng·hr/ml, or 350 ng·hr/ml. In embodiments, the second pharmaceutical composition provides an in vivo plasma profile having a AUC_{0-∞} of less than about, e.g., 300 ng·hr/ml, 250 ng·hr/ml, 200 ng·hr/ml, 150 ng·hr/ml, or 100 ng·hr/ml. In embodiments, the first and second pharmaceutical composition are administered wherein the compositions provide improvement in symptoms in the patient. In embodiments, the first pharmaceutical composition provides improvement in one or more symptoms of cervical dystonia, multiple system atrophy, myoclonus, progressive supranuclear palsy, restless legs syndrome, Wilson's disease, ataxia, chorea, or dystonia for more than, e.g., 6 hours, 8 hours or 12 hours after administration of the first pharmaceutical composition.

In embodiments, the Tₘₐₓ of the first pharmaceutical composition is less than 3 hours. In embodiments, the Tₘₐₓ of the first pharmaceutical composition is less than 2.5 hours. In embodiments, the Tₘₐₓ of the first pharmaceutical composition is less than 2 hours. In embodiments, the Tₘₐₓ of the first pharmaceutical composition is less than 1.5 hours. In embodiments, the Tₘₐₓ of the first pharmaceutical composition is less than 1 hour.

In embodiments, the first pharmaceutical composition provides a dissolution of at least about 80% within the first 20 minutes of administration to a patient in need thereof. In embodiments, the first pharmaceutical composition provides a dissolution of at least about, e.g., 85%, 90% or 95% within the first 20 minutes of administration to a patient in need thereof. In embodiments, the first pharmaceutical composition provides a dissolution of at least 80% within the first 10 minutes of administration to a patient in need thereof.

In embodiments the first and/or the second pharmaceutical compositions are sub therapeutic dosages. A sub therapeutic dosage is an amount of active substance, e.g., gaboxadol, allopregnanolone, or ganaxolone, or a pharmaceutical salt of any of the foregoing, that is less than the amount required for a therapeutic effect. In embodiments, a sub therapeutic dosage is an amount of gaboxadol, allopregnanolone, or ganaxolone, or a pharmaceutical salt of any of the foregoing, that alone may not provide improvement in at least one symptom of cervical dystonia, multiple system atrophy, myoclonus, progressive supranuclear palsy, restless legs syndrome, Wilson's disease, ataxia, chorea, or dystonia, but is sufficient to maintain such improvement. In embodiments, the methods provide administering a first pharmaceutical composition that provides improvement in at least one symptom of cervical dystonia, multiple system atrophy, myoclonus, progressive supranuclear palsy, restless legs syndrome, Wilson's disease, ataxia, chorea, or dystonia, and a second composition that maintains the improvement. In embodiments, after administration of the first pharmaceutical composition, the second pharmaceutical composition may provide a synergistic effect to improve at least one symptom of cervical dystonia, multiple system atrophy, myoclonus, progressive supranuclear palsy, restless legs syndrome, Wilson's disease, ataxia, chorea, or dystonia. In embodiments the second pharmaceutical composition may provide a synergistic effect to improve at least one symptom of an of cervical dystonia, multiple system atrophy, myoclonus, progressive supranuclear palsy, restless legs syndrome, Wilson's disease, ataxia, chorea, or dystonia.

In embodiments, provided herein are methods of treating of cervical dystonia, multiple system atrophy, myoclonus, progressive supranuclear palsy, restless legs syndrome, Wilson's disease, ataxia, chorea, or dystonia, including administering to a patient in need thereof a pharmaceutical composition including a first pharmaceutical dosage wherein the composition provides improvement for more than 6 hours after administration and a second pharmaceutical composition including a sub therapeutic dosage of gaboxadol, allopregnanolone, or ganaxolone, or a pharmaceutical salt of any of the foregoing.

Administration of the first and second pharmaceutical compositions may be simultaneous or separated by an interval of time to achieve immediate, intermediate or long-term improvement in at least one symptom of cervical dystonia, multiple system atrophy, myoclonus, progressive supranuclear palsy, restless legs syndrome, Wilson's disease, ataxia, chorea, or dystonia. In embodiments, the first and second pharmaceutical composition may be administered 6 hours apart. In embodiments, the first and second pharmaceutical composition may be administered 12 hours apart. In embodiments, the first and second pharmaceutical compositions may administered within, e.g., 15 minutes, 30 minutes, 1 hour, 2 hours, 6 hours, 12 hours, 18 hours, 24 hours etc. In embodiments, the first and second pharmaceutical composition may be administered together. In embodiments, the first and second pharmaceutical compositions may administered separated by at least, e.g., 15 minutes, 30 minutes, 1 hour, 2 hours, 12 hours, 18 hours, 24 hours etc. In embodiments, improvement in at least one symptom of cervical dystonia, multiple system atrophy, myoclonus, progressive supranuclear palsy, restless legs syndrome, Wilson's disease, ataxia, chorea, or dystonia for more than 8 hours after administration to the patient is provided. In embodiments, improvement for more than about, e.g., 10 hours, 12 hours, 15 hours, 18 hours, 20 hours, or 24 hours after administration to the patient is provided.

In embodiments, the administration of the first and second pharmaceutical composition may provide a synergistic effect to improve at least one symptom of cervical dystonia, multiple system atrophy, myoclonus, progressive supranuclear palsy, restless legs syndrome, Wilson's disease, ataxia, chorea, or dystonia.

In embodiments, the first and/or the second pharmaceutical composition include any of the aforementioned amounts of active substance, e.g., gaboxadol, allopregnanolone, or ganaxolone, or a pharmaceutical salt of any of the foregoing.

The disclosed compounds, such as gaboxadol, allopregnanolone, or ganaxolone, or a pharmaceutical salt of any of the foregoing, can be used individually as a monotherapy as the only active agent. In embodiments, methods are provided of treating cervical dystonia, multiple system atrophy, myoclonus, progressive supranuclear palsy, restless legs syndrome, Wilson's disease, ataxia, chorea, or dystonia using gaboxadol, allopregnanolone, or ganaxolone, or a pharmaceutical salt of any of the foregoing. In embodiments, methods of treating cervical dystonia, multiple system atrophy, myoclonus, progressive supranuclear palsy, restless legs syndrome, Wilson's disease, ataxia, chorea, or dystonia include administration of gaboxadol, allopregnanolone, or ganaxolone, or a pharmaceutical salt of any of the foregoing in combination with one or more other active agent, e.g., gaboxadol, allopregnanolone, or ganaxolone, or a pharmaceutical salt of any of the foregoing. The combination therapies can include administration of the active agent, e.g., gaboxadol, allopregnanolone, or ganaxolone, or a pharmaceutical salt of any of the foregoing, together in the same admixture, or in separate admixtures. In embodiments, the pharmaceutical composition includes two, three, or more active agents. In embodiments, the combinations result in a more than additive effect on the treatment of the disease or disorder. Thus, treatment is provided of cervical dystonia, multiple system atrophy, myoclonus, progressive supranuclear palsy, restless legs syndrome, Wilson's disease, ataxia, chorea, or dystonia with a combination of agents that combined, may provide a synergistic effect that enhances efficacy.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “treating” or “treatment” refers to alleviating, attenuating or delaying the appearance of clinical symptoms of a disease or condition in a subject that may be afflicted with or predisposed to the disease or condition, but does not yet experience or display clinical or subclinical symptoms of the disease or condition. In certain embodiments, treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” may also refer to inhibiting the disease or condition, e.g., arresting or reducing a movement disorder herein or at least one clinical or subclinical symptom thereof. “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic treatment are two separate embodiments of the disclosure herein.

“Patient in need thereof” may include individuals that have been diagnosed with a movement disorder herein. “Patient” and “subject” are used interchangeably herein.

“Effective amount” or “therapeutically effective amount” means a dosage sufficient to alleviate one or more symptom of a disorder, disease, or condition being treated, or to otherwise provide a desired pharmacological and/or physiologic effect. The “effective amount” or “therapeutically effective amount” can vary depending on the compound, the disease and its severity and the age, weight, physical condition and responsiveness of the subject to be treated. In embodiments, a therapeutically effective amount of active agent(s) is an amount effective to treat a movement disorder herein. The effective amount of the drug for pharmacological action, and therefore the dosage strength may depend on progression of the disease itself “Effective amount” or “therapeutically effective amount” may be used interchangeably herein.

“Improvement” refers to the treatment of symptoms or conditions associated with movement disorders herein, measured relative to at least one symptom or condition of the movement disorder.

“Improvement in next day functioning”, “wherein there is improvement in next day functioning” or “improvement . . . a day after administration” refers to improvement after waking from an overnight sleep period wherein the beneficial effect of administration of one or more of gaboxadol or a pharmaceutically acceptable salt thereof alone, or ganaxolone or a pharmaceutically acceptable salt thereof alone, or allopregnanolone or a pharmaceutically acceptable salt thereof alone, or gaboxadol in combination ganaxolone or allopregnanolone, applies to at least one symptom or condition associated with an movement disorder herein and is discernable, either subjectively by a patient or objectively by an observer, for a period of time, e.g., immediately, 1 hour, 2 hours, hours, 3 hours, 4 hours, 5 hours, 6 hours, 12 hours, 24 hours, etc. after waking.

“Composition”, “pharmaceutical composition”, “formulation”, “pharmaceutical formulation” are used interchangeably herein. “Composition”, “pharmaceutical composition”, “formulation”, “pharmaceutical formulation” encompass dosage forms. Dosage forms can encompass unit doses.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

As used herein, the term “prevention” or “preventing” means to administer a composition to a subject or a system at risk for or having a predisposition for one or more symptoms caused by a disease or disorder to facilitate cessation of a particular symptom of the disease or disorder, a reduction or prevention of one or more symptoms of the disease or disorder, a reduction in the severity of the disease or disorder, the complete ablation of the disease or disorder, stabilization or delay of progression of the disease or disorder.

“Analog” “analogue, and “derivative” are used herein interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

The term “pharmaceutically acceptable salt”, as used herein, refers to derivatives of the compounds defined herein, wherein the parent compound is modified by making acid or base salts thereof.

“Excipient” is a substance, other than the active drug substance of a pharmaceutical composition, which has been appropriately evaluated for safety and are included in a drug delivery system to either aid the processing of the drug delivery system during its manufacture; protect; support; enhance stability, bioavailability, or patient acceptability; assist in product identification; or enhance any other attributes of the overall safety and effectiveness of the drug delivery system during storage or use.

“Stabilizer” or “stabilizing amount” refers to an amount of one or more excipients included in the parenteral compositions that provide sufficient stability but do not adversely affect the bioavailability, safety and/or efficacy of gaboxadol, allopregnanolone, or ganaxolone, or a pharmaceutical salt of any of the foregoing used in the composition.

“Stable” means that there is substantially no degradation of the gaboxadol, allopregnanolone, or ganaxolone, or a pharmaceutical salt of any of the foregoing, after a specified period of time, e.g., after 3 months or 6 months.

“Soluble” means that the solution of al gaboxadol, allopregnanolone, or ganaxolone, or a pharmaceutical salt of any of the foregoing does not turn hazy and/or there is substantially no precipitate in the solution.

“Sufficiently soluble” means that the particle content is sufficiently low, and the material is sufficiently sterile such that it is useful for parenteral administration. For example, the number of particles in a liquid composition should be, e.g., less than 6,000 10 μm particles should be present in a volume of 10 ml solvent, preferably less than 10,000, less than 5,000, less than 3,000, less than 1,000, or less than 400 10 μm particles. In some examples, the number of particles in a liquid composition should be less than 1000, less than 600, or less than 200 25 μm particles in the 10 ml volume.

“Local site compatible” herein shall mean the composition is tolerant at the site of injection or infusion, thus minimizing side effects, such as local skin irritations or venous irritations, including inflammatory reactions at the infusion site. The parenteral compositions herein may have less side reactions than conventional products, such as skin irritation or phlebitis.

“Purified” as used herein refers to material that has been isolated under conditions that reduce or eliminate the presence of unrelated materials, i.e., contaminants, including native materials from which the material is obtained. As used herein, the term “substantially free” is used operationally, in the context of analytical testing of the material. Preferably, purified material substantially free of contaminants is at least 95% pure; more preferably, at least 97% pure, and more preferably still at least 99% pure. Purity can be evaluated, for example, by chromatography or any other methods known in the art. In embodiments, purified means that the level of contaminants is below a level acceptable to regulatory authorities for safe administration to a human or non-human animal.

“Ready-to-use” with reference to the compositions herein shall mean the preparation in the reconstituted form, with standardized concentration and quality, prefilled in the single-use container, such as glass vials, infusion bags or syringes, ready for direct administration to the patient.

“Direct administration” with reference to the compositions herein shall mean the immediate administration, i.e., without further dilution, premixing with other substances or otherwise changing the composition or formulation of the composition. Such composition is typically directly discharged from an infusion device and administered via a vascular access port or through a central line.

“Dosage” is intended to encompass a formulation expressed in terms of μg/kg/day, μg/kg/hr, mg/kg/day or mg/kg/hr. The dosage is the amount of an ingredient administered in accordance with a particular dosage regimen. A “dose” is an amount of an agent administered to a mammal in a unit volume or mass, e.g., an absolute unit dose expressed in mg or μg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning in any case will be apparent from context.

“Co-administered with”, “in combination with”, “administered in combination with”, “a combination of”, “administered along with”, or “co-therapy”, may be used interchangeably and mean that two or more agents are administered in the course of therapy. The agents may be administered together at the same time or separately in spaced apart intervals. The agents may be administered in a single dosage form or in separate dosage forms.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml or μg·hr/ml). The area under the curve is governed by clearance. Clearance is defined as the volume of blood or plasma that is totally cleared of its content of drug per unit time (ml/min).

### EXAMPLES

The Examples provided herein are included solely for augmenting the disclosure herein and should not be considered to be limiting in any respect.

### Example 1

Gaboxadol Plasma Concentration Profiles

The following Example provides the plasma concentration profiles and dose proportionality of gaboxadol monohydrate following single oral doses ranging from 2.5 to 20 mg. The absolute bioavailability of gaboxadol monohydrate capsules ranging from 2.5 to 20 mg is also assessed.

This study was composed of separate groups of 10 healthy adult subjects (at least 4 of each gender) who participated in a 6-period, double-blind, randomized, crossover study designed to access the dose proportionality and absolute bioavailabilty of 5 single oral doses of gaboxadol across the dose range of 2.5 to 20 mg. The order in which the subjects received the 5 single oral doses of gaboxadol (2.5; 5; 10; 15; and 20 mg) was randomized within Treatment Periods 1 through 5. Each subject was expected to complete all 6 treatment periods and there was a washout of at least 4 days between each treatment period.

Each oral dosing within Treatment Periods consisted of 2 capsules of test drug taken simultaneously at each scheduled dosing. The treatment designations for the orally administered study drugs were as follows: Treatment A—one 2.5 mg gaboxadol capsule and 1 matching placebo capsule; Treatment B—one 5 mg gaboxadol capsule and 1 matching placebo capsule; Treatment C—one 10 mg gaboxadol capsule and 1 matching placebo capsule; Treatment D—one 15 mg gaboxadol capsule and 1 matching placebo capsule; and Treatment E—20 mg gaboxadol (two 10 mg gaboxadol capsules). Subjects received their study drug after an overnight fast with 240 mL of water in the morning about 8:00 AM. Water was permitted ad libitum except within 1 hour prior to and after study drug administration. No food was allowed for 4 hours post dose.

For each subject in each treatment, plasma and urine samples were collected over 16 hours post-dosing for the determination of pharmacokinetic parameters (e.g., AUC, Cₘₐₓ, Tₘₐₓ, apparent t_{1/2}, cumulative urinary excretion, renal clearance, clearance, and steady-state volume of distribution, as appropriate). AUC and Cₘₐₓ for gaboxadol were potency adjusted to facilitate comparison of pharmacokinetic data across studies. Table 1 provides the individual potency-adjusted pharmacokinetic parameters of gaboxadol following single oral doses (2.5, 5, 10, 15, and 20 mg).

TABLE 1
Pharmacokinetic parameters for gaboxadol following oral and IV administration
Pharmacokinetic parameters for gaboxadol following oral and IV administration

| | Geometric Mean (N = 10) | | | | | | |
|---|---|---|---|---|---|---|---|
| | | | 10 | 10 | | | |
| | 2.5 | 5 | mg | mg | 15 | 20 | |
| Parameter | mg | mg | Oral | I.V. | mg | mg | Slope (90% CI) ^{††} |
| AUC_{0-∞} | 90 | 171 | 346 | 380 | 539 | 669 | 0.98 (0.95, 1.01) |
| (ng · hr/mL) | | | | | | | |
| Cₘₐₓ (ng/mL)^{†} | 61 | 110 | 232 | 212 | 382 | 393 | 0.95 (0.88, 1.02) |
| Tₘₐₓ (hr)^{‡} | 0.5 | 0.6 | 0.5 | — | 0.5 | 0.6 | |
| Apparent t_{1/2} (hr)^{§} | 1.5 | 1.5 | 1.6 | 1.5 | 1.5 | 1.6 | |
| CL/F (mL/min)^{ϑ} | 461 | 488 | 476 | 438 | 469 | 499 | |
| Fₑ (%) | 43 | 45 | 53 | 53 | 50 | 53 | |
| CL_{R} (mL/min) | 196 | 222 | 250 | 208 | 234 | 265 | |

| | |
|---|---|
| F (%) (90% CI)^{#} | 92% (0.86, 0.97) |

^{†}C_{coi} (ng/mL) for 10 mg. IV.
^{‡}Median.
^{§}Harmonic Mean.
^{ϑ}CL (mL/min) for 10 mg IV.
^{#}Bioavailability relative to 10 mg I.V. reference based on pooled dose-adjusted (to 10 mg) oral AUC_{0-∞} values.
^{††} Dose proportionality assessment of oral treatments only.

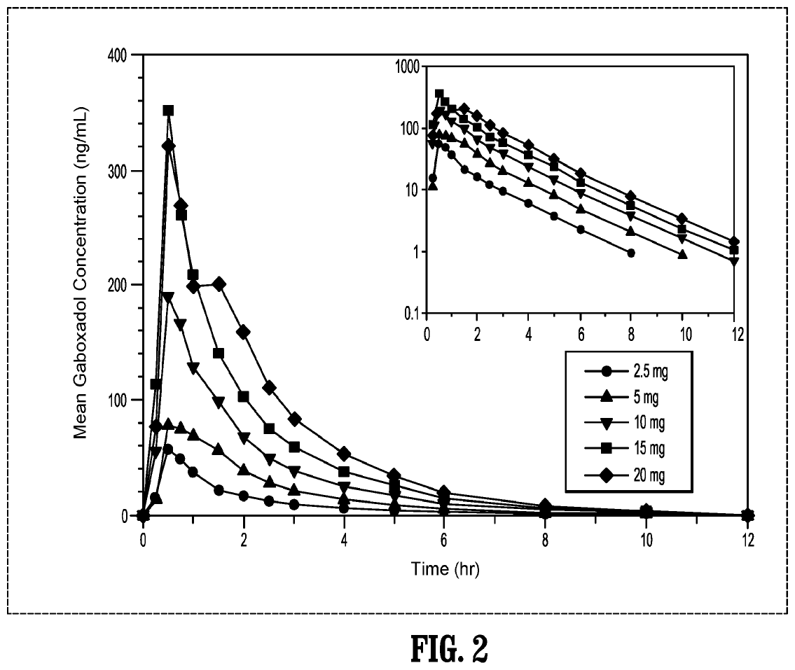

FIG. 1 shows the arithmetic mean plasma concentration-time profiles of gaboxadol following single oral doses (2.5, 5, 10, 15, and 20 mg) as described in Example 1 with horizontal lines Δ indicating the change between 6 and 12 hours. FIG. 2 shows the arithmetic mean plasma concentration-time profiles of gaboxadol following single oral doses (2.5, 5, 10, 15, and 20 mg).

### Example 2

Assessment of Residual Effects Resulting from Gaboxadol Administration

This study was a double blind, double-dummy, randomized, active- and placebo-controlled, single dose, 3-period crossover study, followed by an open-label, single-dose, single period study in healthy elderly male and female subjects. Subjects were randomized to each of 3 treatments (Treatments A, B, and C) to be administered in a crossover manner over the first 3 treatment periods. For Treatment A, subjects received a single dose of gaboxadol 10 mg; for Treatment B, subjects received a single dose of flurazepam 30 mg; and for Treatment C, subjects received a single dose of placebo. Doses were administered orally at bedtime on Day 1. Subjects were domiciled from early in the evening of dosing until ˜36 hours post-dose (morning of Day 3) during each treatment period. The subjects who participated in treatment periods 1-3 participated in a fourth treatment period. In this period, a single dose of gaboxadol 10 mg (Treatment D) was administered orally in an open-label manner on the morning of Day 1 for PK of gaboxadol. There was at least a 14-day washout between the doses of consecutive treatment periods. Study participants included healthy, elderly male and female subjects between 65 and 80 years of age, with a Mini Mental Status 24, weighing at least 55 kg. All subjects received 10 mg gaboxadol monohydrate capsules and 30 mg flurazepam (provided as 2×15 mg capsules), matching placebo was provided for both gaboxadol and flurazepam.

The primary endpoints evaluated included pharmacodynamics (measurement of psychomotor performance, memory, attention and daytime sleepiness the following pm dosing), gaboxadol pharmacokinetics, and safety. Gaboxadol (single dose 10 mg) did not show residual effect 9 hours post-dose on the primary endpoints Choice Reaction Time and Critical Flicker Fusion, whereas the active reference Flurazepam (30 mg single dose) showed significant effect on the same tests. In addition, gaboxadol did not show any signs of residual effects on other measurements applied in the study (Multiple Sleep Latency Test (MSLT); Digit symbol substitution test (DSST), Tracking, Memory tests, Body Sway, and Leeds Sleep Evaluation Questionnaire).

Those skilled in the art will recognize, or be able to ascertain using no more than routine experimentation, many equivalents to the specific embodiments described herein. Such equivalents are intended to be encompassed by the claims.

What is claimed is:

1. A method for treating multiple system atrophy comprising administering to a patient in need thereof about 0.05 mg to about 30 mg gaboxadol or a pharmaceutically acceptable salt thereof.

2. The method of claim 1, wherein the method provides improvement in one or more symptoms of the multiple system atrophy in the patient.

3. The method of claim 2, wherein the improvement is provided for more than 6 hours after administration.

4. The method of claim 1, wherein the patient is administered a composition comprising about 1 mg to about 15 mg gaboxadol or a pharmaceutically acceptable salt thereof.

5. The method of claim 1, wherein the in vivo plasma profile of the patient 6 hours after administration of the gaboxadol or pharmaceutically acceptable salt thereof is reduced by more than 50%.

6. The method of claim 1, wherein the AUC₆₋₁₂ of the patient 6 hours after administration of the gaboxadol or pharmaceutically acceptable salt thereof is less than 75% of the administered dose.

7. The method of claim 2, wherein the composition provides improvement in the patient for more than 12 hours after the administering.
